SEQUENCE LISTING (2 sequences)

SEQ ID NO: 1 - DNA, 2552 bp, Homo sapiens
  ref <308>: DATABASE ACCESSION NUMBER: NCBI NM_001277204.1
  ref <309>: DATABASE ENTRY DATE: 2015-03-15
  ref <313>: RELEVANT RESIDUES IN SEQ ID NO: (1)..(2552)
gtttgagtgagcaaacagagggtgaatgaagtgacttgcgggcagctgggtagaggatgagtcagcattggtgtaaaggactggagcttcagggttctcgggattcgagtcctggccctgctgcttgccagctctgtgctgtggcaagttacttcctctccccaggtctctcggtttcctcatctgctgcctctccagacttctgccagaacattgcacgcgacagtttcaggcacagaactgactggcagcaggggctgctccacgagtgggaatttgctccagcacttcacggactgcaagcgaggcacttgctaactcttggataacaagacctctgccagaagaaccatggctttggaaggcggagttcaggctgaggagatgggtgcggtcctcagtgagcccctgcctccctgaacataggaaacccacctgggcagccatggaatgggacaatggcacaggccaggctctgggcttgccacccaccacctgtgtctaccgcgagaacttcaagcaactgctgctgccacctgtgtattcggcggtgctggcggctggcctgccgctgaacatctgtgtcattacccagatctgcacgtcccgccgggccctgacccgcacggccgtgtacaccctaaaccttgctctggctgacctgctatatgcctgctccctgcccctgctcatctacaactatgcccaaggtgatcactggccctttggcgacttcgcctgccgcctggtccgcttcctcttctatgccaacctgcacggcagcatcctcttcctcacctgcatcagcttccagcgctacctgggcatctgccacccgctggccccctggcacaaacgtgggggccgccgggctgcctggctagtgtgtgtagccgtgtggctggccgtgacaacccagtgcctgcccacagccatcttcgctgccacaggcatccagcgtaaccgcactgtctgctatgacctcagcccgcctgccctggccacccactatatgccctatggcatggctctcactgtcatcggcttcctgctgccctttgctgccctgctggcctgctactgtctcctggcctgccgcctgtgccgccaggatggcccggcagagcctgtggcccaggagcggcgtggcaaggcggcccgcatggccgtggtggtggctgctgcctttgccatcagcttcctgccttttcacatcaccaagacagcctacctggcagtgcgctcgacgccgggcgtcccctgcactgtattggaggcctttgcagcggcctacaaaggcacgcggccgtttgccagtgccaacagcgtgctggaccccatcctcttctacttcacccagaagaagttccgccggcgaccacatgagctcctacagaaactcacagccaaatggcagaggcagggtcgctgagtcctccaggtcctgggcagccttcatatttgccattgtgtccggggcaccaggagccccaccaaccccaaaccatgcggagaattagagttcagctcagctgggcatggagttaagatccctcacaggacccagaagctcaccaaaaactatttcttcagccccttctctggcccagaccctgtgggcatggagatggacagacctgggcctggctcttgagaggtcccagtcagccatggagagctggggaaaccacattaaggtgctcacaaaaatacagtgtgacgtgtactgtcatcaaggggtatgctccatgctttgagtcaccaatgaagcgggtgagggaagatgagagggggaggtgagagcttctgggaaggggcatttgagctgggttttgagggatgattatgagctctctggagaggagtgatattccatttatttagaaagcctttactgacaccttgtgctcaggcctgtgttggttctggggccctagaagaaccagtcctagccctggtccatacgggctcccaactgctggaagtacagactggcacagcaacatcagggttgtgacagagggaagcatggctggggggagggggtacacagacagtgcccatgacccagtccaaggagtcaggaaagagctctctgaggagggagcatctgagccagatgttgagggctgagtgggaacttggcaagcagaagtggggagcactttaatgcaacccaggtatgctccatgcatatccagctgggccagcctcgtgctgggctctgccctgggcagacaggcagagggccagagcagaggacacatggccttgcgtgtgtgaaagctgagaaaatgggagctgtgcttcagctaccctccagacaagggcaagagttagccagatgctccaggcagtgggaagccaatggagggattaagcggcggaagcttcttagaagaggcagggggcgaagtgcagtggctcatgcctgtaatctcagcaatttgggaggccaaggaaggaggaatgcttgagcccaagagttttagaccagcctgggcaacacagtaagaccctgtttctacaaaaaaatataaaaaatagcctgg

SEQ ID NO: 2 - protein, 328 aa, Homo sapiens
  ref <308>: DATABASE ACCESSION NUMBER: NCBI NP_001264133
  ref <309>: DATABASE ENTRY DATE: 2015-03-15
  ref <313>: RELEVANT RESIDUES IN SEQ ID NO: (1)..(328)
MEWDNGTGQALGLPPTTCVYRENFKQLLLPPVYSAVLAAGLPLNICVITQICTSRRALTRTAVYTLNLALADLLYACSLPLLIYNYAQGDHWPFGDFACRLVRFLFYANLHGSILFLTCISFQRYLGICHPLAPWHKRGGRRAAWLVCVAVWLAVTTQCLPTAIFAATGIQRNRTVCYDLSPPALATHYMPYGMALTVIGFLLPFAALLACYCLLACRLCRQDGPAEPVAQERRGKAARMAVVVAAAFAISFLPFHITKTAYLAVRSTPGVPCTVLEAFAAAYKGTRPFASANSVLDPILFYFTQKKFRRRPHELLQKLTAKWQRQGR

(19) United States
(12) Patent Application Publication
(10) Pub. No.: US 2018/0325933 A1
(43) Pub. Date: Nov. 15, 2018

(54) FOOD INTAKE, BODY WEIGHT AND GLUCOSE METABOLISM REGULATION BY MODULATION OF P2Y6 RECEPTOR SIGNALING

(71) Applicant: Max-Planck-Gesellschaft zur Förderung der Wissenschaften e.V., Munich (DE)

(72) Inventors: Jens Brüning, Köln (DE); Sophie Steculorum, Köln (DE)

(21) Appl. No.: 15/326,562

(22) PCT Filed: Apr. 14, 2015
(86) PCT No.: PCT/EP2015/058114

§ 371 Date: Jan. 16, 2017

(30) Foreign Application Priority Data
Jul. 16, 2014 (EP) 14177351.5
Sep. 30, 2014 (EP) 14187205.1

(51) Int. Cl. A61K 31/7072; A61P 3/08; G01N 33/566

(52) U.S. Cl. CPC A61K 31/7072; A61P 3/08; G01N 2800/042; G01N 2333/726; G01N 2500/04; G01N 33/566

(57) ABSTRACT

The present invention is related to compound capable of regulating the activity of P2Y purinoceptor 6 signaling pathway, especially to compounds for inhibition of P2Y purinoceptor 6 polypeptide or inactivation, degradation, downregulation or intercalation of a nucleic acid encoding P2Y purinoceptor 6 or downregulation of P2Y purinoceptor 6 signaling pathway for the treatment of diseases related to energy balance as well as carbohydrate metabolism and homeostasis, preferably glucose metabolism and homeostasis. The present is also related to compounds for activation of P2Y purinoceptor 6 polypeptide or upregulation or modification for advanced transcriptional activity of a nucleic acid encoding P2Y purinoceptor 6, upregulation of P2Y purinoceptor 6 signaling pathway for gaining weight or for the treatment of diseases related to energy balance and carbohydrate metabolism and homeostasis. The invention is further related to methods of identifying said compounds suitable for the treatment of diseases related to energy balance and carbohydrate metabolism and homeostasis. The invention is further related to methods of treatment and diagnosis of diseases related to energy balance and carbohydrate metabolism and homeostasis and associated complications.

The present invention is related to compounds capable of regulating the activity of P2Y purinoceptor 6 signaling pathway, especially to compounds for inhibition of P2Y purinoceptor 6 polypeptide or inactivation, degradation, downregulation or intercalation of a nucleic acid encoding P2Y purinoceptor 6 or downregulation of P2Y purinoceptor 6 signaling pathway for the treatment of diseases related to energy balance as well as carbohydrate metabolism and homeostasis, preferably glucose metabolism and homeostasis, such as obesity, type 2 diabetes mellitus (T2D), and related complications selected from cardiovascular diseases, hepatic steatosis and lipid disorders. The present is also related to compounds for activation of P2Y purinoceptor 6 polypeptide or upregulation or modification for advanced transcriptional activity of a nucleic acid encoding P2Y purinoceptor 6, upregulation of P2Y purinoceptor 6 signaling pathway for gaining or maintaining weight, such as anorexia nervosa and cancer cachexia.

The invention is further related to methods of identifying said compounds suitable for the treatment of diseases related to energy balance and carbohydrate metabolism and homeostasis. The invention is further related to methods of treatment and diagnosis of diseases related to energy balance and carbohydrate metabolism and homeostasis, such as obesity, type 2 diabetes mellitus (T2D), and related complications (related to obesity and/or type 2 diabetes) selected from cardiovascular diseases, hepatic steatosis and lipid disorders.

Diabetes as a leading cause of death in developed countries is a metabolic condition characterized by high blood sugar levels. There are two main types of diabetes: type 1, resulting from insufficient insulin production of the pancreatic beta cells, which requires the person to inject insulin; and type 2, resulting from insensitivity of peripheral tissues to insulin (such skeletal muscle, liver or adipose tissue), insulin release alterations, and relative insulin deficiency.

Type 1 diabetes is a genetic or autoimmune disease; the only effective therapy to date is the supply of exogenous insulin. This therapy does not cure type 1 diabetes; the person needs continuous supply of insulin.

The decreased insulin sensitivity of peripheral tissues in T2D that accounts for 90% of all cases of the disease is initially compensated by an increased release of insulin by the beta cells of the pancreas. At a certain stage of the disease, the pancreas cannot maintain the increases release of insulin anymore. T2D is often acquired and accompanied by obesity; it can be treated in first hand by reducing weight, diet and exercise. Patients diagnosed with T2D often require pharmaceutical medications to control their symptoms. Obesity is defined as abnormal or excessive fat accumulation that may impair health. The body mass index (BMI; weight in kg divided by height in meters squared) may be used to classify overweight and obesity. BMI are sex and age dependent. In general overweight is defined as having a BMI≥25 kg/m² and obesity is defined as having a BMI≥30 kg/m².

In the midst of the present escalating prevalence of obesity and T2D, there is an urgent need for unraveling the exact mechanisms underlying the control of body weight and glucose homeostasis. Researches led during the past decades pinpointed the critical importance of the central nervous system (CNS), and more particularly the hypothalamus, in homeostatic processes governing energy balance and glycemic control. Particular attention has been paid to the pivotal role of the arcuate nucleus of the hypothalamus (ARH), which contains two main antagonistic neuronal populations: the orexigenic neurons co-expressing neuropeptide Y (NPY) and agouti-related peptide (AgRP) and the anorexigenic neurons that synthesize proopiomelanocortin (POMC). These two populations of neurons are well described for being direct targets of the adipoctyte-derived hormone leptin and the pancreatic hormone insulin, both of which are anorexigenic and secreted proportionally to body fat mass. The discovery that obesity and T2D are associated with the onset of neuronal leptin and insulin resistance complicates the understanding of these ARH neuronal circuitries and largely limits pharmaceutical interventions targeting these hormonal pathways.

The inventors identified that a G-protein coupled receptor, the purinergic receptor 6 (synonymously called P2Y purinoceptor 6 or P2Y₆), is highly expressed in the hypothalamus. It was found that P2Y₆ displays a specific regional pattern of expression and is particularly highly expressed in the ARH. P2Y₆ is part of the purinergic receptor family that contains some of the most abundant receptors in living organisms and is highly conserved throughout evolution. The metabotropic P2Y₆ receptors are G protein-coupled receptors and are mainly responsive to uridine diphosphate (UDP), for which they are the sole receptors.

The inventors further explored P2Y₆ and its ligands, one of those is UDP, and found that P2Y₆/UDP pathway is involved in metabolic regulation and therefore can control the onset and the progression of diseases related to energy balance and carbohydrate metabolism and homeostasis such as obesity and diabetes. In particular, it is proposed that P2Y₆ antagonism inhibits eating or food intake and thus is suitable for treatment of obesity.

The objective of the present invention is to provide targets for the treatment of diseases related to energy balance and carbohydrate metabolism and homeostasis and related complications. This goal is achieved by the claimed compounds, which regulate the activity of P2Y purinoceptor 6 signaling pathway. Further advantageous embodiments, aspects and details of the invention are evident from the depending claims, the description, the examples and the figures.

### SHORT DESCRIPTION

The invention refers particularly to a compound for use in therapy of diseases related to energy balance as well as carbohydrate metabolism and homeostasis and complications associated, wherein the compound preferably regulates hypothalamic level of UDP. That compound may be a regulator of uridine transport to the CNS (transport over the blood brain barrier) or a regulator of the synthesis, respectively the enzymes important for the synthesis of UDP in the CNS. In the CNS only a low level of de novo pyrimidine synthesis has been reported. Most of the pyrimidine (including uridine) content in the CNS is supplied by the uptake of pyrimidine nucleosides. Uridine-phosphorylating enzymes (uridine kinase and UMP kinase) are of low affinity. Consequently, providing the CNS with uridine increases formation of UDP and thereby UDP levels. Thus, regulation of uridine transport to the CNS is a promising target for influencing UDP levels in the CNS and consequently regulating P2Y purinoceptor 6 signaling pathway. Receptors transporting uridine and being responsible for the transport of uridine into the brain are part of the family of concentrative nucleoside transporters (CNT 1-3; Na+-nucleoside transporter) and equilibrative nucleoside transporters (ENT 1-4).

The invention refers further to a method for screening for a compound for treatment of diseases related to energy balance and carbohydrate metabolism and homeostasis, wherein the method comprises providing a test compound for contacting at least one P2Y₆ polypeptide or nucleic acid coding for P2Y₆, detecting the binding of said test compound to the P2Y₆ polypeptide or nucleic acid coding for P2Y₆ polypeptide, and determining the activity of the P2Y₆ polypeptide in the presence of said test compound. The invention refers also to a compound that regulates the activity of P2Y purinoceptor 6 signaling pathway. In a preferred embodiment of the invention said compound is suitable for inhibition of P2Y purinoceptor 6 polypeptide or for inactivation, degradation, downregulation or intercalation of a nucleic acid encoding P2Y purinoceptor 6 for the treatment of a disease related to energy balance and carbohydrate metabolism and homeostasis.

Said compound can be chosen from the group comprising a small molecule, an RNA molecule, a siRNA molecule, a miRNA molecule, or a precursor thereof, an antisense oligonucleotide, an aptamer, a polypeptide, an antibody, or a ribozyme, wherein RNA, peptides, small molecules and aptamers are preferred compounds.

The invention refers further to a pharmaceutical composition comprising a compound for the treatment of diseases related to energy balance and carbohydrate metabolism and homeostasis, more preferred of obesity or diabetes, more preferred of diabetes mellitus type 2, and related complications selected from cardiovascular diseases, hepatic steatosis and lipid disorders.

The invention further provides a method for treatment of diseases related to energy balance and carbohydrate metabolism and homeostasis comprising administering a subject in need thereof a therapeutically effective amount of at least one compound for inhibition of P2Y purinoceptor 6 polypeptide or for inactivation, degradation, downregulation or intercalation of a nucleic acid encoding P2Y purinoceptor 6.

The present invention refers further to a compound for
a) activation of P2Y purinoceptor 6 polypeptide,
b) upregulation or modification for advanced transcriptional activity of a nucleic acid encoding P2Y purinoceptor 6,
c) upregulation of P2Y purinoceptor 6 signaling pathway,
d) activation of uridine transport across the blood brain barrier,
e) activation or increase of UDP synthesis in the CNS for use in a therapy for gaining weight.

Thereby it is preferred that the hypothalamic levels of UDP are increased for gaining weight. In another preferred embodiment of the invention said compound is suitable for activation of P2Y purinoceptor 6 polypeptide or for activation, or upregulation of a nucleic acid encoding P2Y purinoceptor 6 for the treatment of a disease related to energy balance and carbohydrate metabolism and homeostasis, in particular for use in a therapy gaining or maintaining weight which is desirable for the treatment of diseases such as anorexia nervosa or cancer cachexia.

The invention further provides a method for treatment of cancer cachexia, underweight, especially treatment of newborns being underweight, treatment of people in extreme need of care being underweight comprising administering a subject in need thereof a therapeutically effective amount of at least one compound for activation of P2Y purinoceptor 6 polypeptide or for activation, upregulation of a nucleic acid encoding P2Y purinoceptor 6.

### DESCRIPTION

WO 2004106937 A2 relates to P2Y₆ and its regulation for the treatment of cardiovascular disorders, cancer and some other diseases. Disease of carbohydrate metabolism and homeostasis such as obesity and diabetes are only mentioned as risk factors of cardiovascular disease but it is not disclosed that regulation of P2Y₆ is suitable for the treatment of these diseases. WO 2004106937 A2 discloses also a screening method for compounds having P2Y₆ as a target. But again it is not suggested that these method may also be appropriate to screen for compounds suitable for treatment of diabetes or obesity or regulation of body weight.

Ramachandran Balasubramanian et al. (Biochemical Pharmacology 2009) disclose the effect of P2Y receptors on insulin secretion in the presence of glucose and teache an increase of insulin secretion after P2Y6 agonist application. Thereby activation of P2Y receptors by specific agonists results in enhanced insulin secretion in MIN6 mouse pancreatic beta cell line. But the present invention refers to administration of P2Y₆ antagonist for treatment of diseases related to carbohydrate metabolism, such as obesity and diabetes; preferably type 2 diabetes mellitus, using their effect in the CNS regulating the CNS-dependent control of energy and glucose homeostasis.

The inventors of JP 2000319185 A showed that food containing uridine or a prodrug of uridine has impact on the blood lipid levels, especially on the level of HDL and LDL. On basis of these results they expect uridine to be useful in the prevention and improvement of obesity. Nevertheless, there is no experimental proof that UDP administration has impact on foot intake, fat mass or body weight (cf. FIG. 11 where no significant difference is shown). Also JP 2007070253 A discloses that uridylic acid and uridine have an anti-obesity activity or anti-diabetic activity. The only example shows an increase in leptin, glucose as well as insulin concentration in rats' plasma (n=10) after administration of uridine. US 2004121979 A1 discloses the use of uridine ester (uridine esterified with fatty acids at the 5′ position of the ribose or deoxyribose moiety) for the treatment of diabetes or obesity.

Opposing these disclosures, it was surprisingly found that UDP plays a direct role on the central regulation of feeding behavior, shown as effect of intracerebroventricular administration (ICV) of UDP on spontaneous food intake in wild type mice fed a normal chow diet. ICV administration of UDP increases food intake in a dose-dependent manner (FIG. 1A). ICV administration of a non-competitive selective antagonist of P2Y₆ decreases food intake (FIG. 1B). This set of data shows that central UDP and P2Y₆ modulate feeding behavior and that inhibition of P2Y₆ is able to decrease food intake. Furthermore, the data shows that P2Y₆ is particularly enriched in the arcuate nucleus (FIG. 2A). The expression levels of P2Y₆-mRNA in different hypothalamic regions such as the ARH, the paraventricular nucleus of the hypothalamus (PVH), the ventromedial nucleus of the hypothalamus (VMH), the dorsomedial nucleus of the hypothalamus (DMH) and the lateral hypothalamic area (LHA) were compared. The greatest mRNA-expression level of P2Y₆ was found in the ARH, where it is expressed=30 to 50% higher than in other hypothalamic regions (FIG. 2A). Furthermore, the expression levels of uridine-cytidine kinase 1 and 2 (UCK-1 and -2), which are the rate limiting enzymes for UDP synthesis, are also high in the ARH (FIGS. 2B and 2C).

Furthermore, the inventors could show that UDP directly activates signaling of ARH via activation of P2Y₆, which is in perfect accordance with its ability to promote feeding (FIGS. 3A and 3B). In addition, the inventors found that UDP increases action potential frequency of AgRP neurons (FIG. 4 B-E). Surprisingly, the inventors discovered that UDP/P2Y₆ are new players in the central modulation of food intake and pinpointed a novel pathway controlling NPY/AgRP neurons. Data presented here reveals that UDP/P2Y₆ signaling in the brain is strongly involved in feeding regulation and that abnormal UDP levels might contribute to the development of obesity and T2D. Furthermore, it is shown that centrally applied UDP enhances food intake, and that this effect is abolished, when activation of AgRP-cells is specifically inhibited (FIGS. 5A and 5B). Additionally, it is clearly demonstrated that UDP specifically engages P2Y₆-signaling for increasing food intake (FIG. 6A) as well as action potential frequency (FIG. 6 C-D). Accordingly, UDP's ability to increase feeding is specifically mediated via P2Y₆-dependent signal transduction. Finally, the inventors could prove that under conditions of obesity, the levels of expression of P2Y₆ in the hypothalamus are unchanged (FIGS. 7 A and B), while hypothalamic concentrations of the P2Y₆ ligand UDP are significantly increased in the absence of alterations in P2Y₆ mRNA-expression (FIGS. 7C and D). When in obesity circulating uridine concentrations are increased (FIG. 8A), this provides enhanced substrate availability of hypothalamic UDP-synthesis, then ultimately feeding is promoted via UDP-induced P2Y₆ signaling in the CNS. In this line, the discovery that pharmacological inhibition of P2Y₆ decreases food intake suggests putative value in cases of hyperphagic obesity.

It is shown that UDP directly activates AgRP-neurons in the ARH in vitro and in vivo (FIG. 3C and FIG. 4A). AgRP-neurons are also known for acting as a key player in the brain-liver axis as they directly controls hepatic gluconeogenesis. Silencing of these neurons leads to suppression of hepatic glucose production (Konner et al., Cell Metabolism 5, 438-449, 2007). Decreasing production of glucose, primarily in the liver, is one of the main physiological effects of insulin, which is directly useful in reducing high blood glucose levels. Based on the UDP-induced activation of NPY/AgRP neurons and the well described role of this neurons in the maintenance of euglycemia, antagonism of P2Y₆ will not only be beneficial to regulate feeding behavior but also to control alteration of glucose homeostasis, such as occur in T2D.

According to the invention, regulation of hypothalamic UDP level and P2Y purinoceptor 6 receptor pathway in the brain regulate caloric intake and are thus preferred targets for a therapy of obesity and diabetes, preferably diabetes type 2, and associated diseases. Therefore the present invention refers to a compound that regulates the activity of P2Y purinoceptor 6 signaling pathway for use in the treatment of diseases related to energy balance, carbohydrate metabolism and homeostasis. As used herein the term “diseases related to energy balance and carbohydrate metabolism and homeostasis” refers preferably to obesity and diabetes, wherein diabetes is preferably diabetes mellitus type 2, and most preferably therapeutic control of insulin resistance or insensitivity in type 2 diabetes mellitus.

The present invention refers particularly to a compound for
a) inhibition of P2Y purinoceptor 6 polypeptide or
b) inactivation, degradation, downregulation or intercalation of a nucleic acid encoding P2Y purinoceptor 6,
c) downregulation of P2Y purinoceptor 6 signaling pathway
d) inhibition of uridine transport across the blood brain barrier,
e) inhibition of UDP synthesis in the CNS,
f) acceleration or increase of UDP degradation

for use in the treatment of diseases related to energy balance and carbohydrate metabolism and homeostasis, preferably including obesity and type 2 diabetes and associated diseases.

In a further embodiment refers the invention to a compound for
a) inhibition of P2Y purinoceptor 6 polypeptide or
b) inactivation, degradation, downregulation or intercalation of a nucleic acid encoding P2Y purinoceptor 6,
c) downregulation of P2Y purinoceptor 6 signaling pathway
d) inhibition of uridine transport across the blood brain barrier,
e) inhibition of UDP synthesis in the CNS,
f) acceleration or increase of UDP degradation

for use in the treatment of diseases caused by deregulated eating or food intake, preferably caused by increased eating or food intake.

Thus, in a preferred embodiment of the invention, the action of the receptor P2Y₆ is blocked by an inhibitor or even more preferred by an antagonist for use in the treatment of obesity and type 2 diabetes and associated diseases. In another preferred embodiment of the invention the hypothalamic levels of UDP are decreased for use in the treatment of obesity and type 2 diabetes and associated diseases.

Hence, one preferred embodiment of the present invention refers to an inhibitor of P2Y purinoceptor 6 polypeptide for the treatment of a disease related to energy balance as well carbohydrate homeostasis and metabolism, notably obesity and type 2 diabetes and associated complications.

It is sufficient to block activity of P2Y₆. However, inhibition of uridine transport over the blood brain barrier, the UDP synthesis or activation of UDP degradation in the CNS may exert a similar therapeutic effect. Receptor inhibitors are, in general, molecules, which bind to receptors and decrease their downstream signaling. The binding of an inhibitor can stop a natural ligand, such as UDP, from entering its binding site, for example by binding the receptor and causing a conformational change of the receptor in a way that the ligand cannot bind. Alternatively, the inhibitor may compete with the natural ligand for the binding site, or hinder the receptor from its reaction to ligand binding, e.g. an enzymatic reaction or a conformational change. Inhibitor binding can be reversible or irreversible. Typical inhibitors of a receptor are antagonist.

A compound according to the present invention can be a molecule which interacts directly or indirectly with the target polypeptide, such as P2Y₆, wherein, as a consequence of the interaction, the activity, e.g. the downstream signaling pathway is inhibited, blocked or has decreased activity. An inhibition of P2Y₆ may be reversible or irreversible or may be competitive or allosteric.

A compound according to the invention may interact with the P2Y₆ polypeptide, e.g. by binding the P2Y₆ polypeptide in a manner leading to conformational changes, masking, binding and/or degradation of the target. Compared to an activity level observed in untreated cells or organism, a decreased signaling activity means a change or decrease in the activity of so called second messenger downstream of P2Y₆ and/or downstream effectors by at least factor 1.5, preferably by factor 2, more preferably by factor 5.

If the target structure is a nucleic acid such as DNA or RNA, such as P2Y₆ encoding DNA or mRNA, a compound of the invention can be a molecule interacting directly or indirectly, e.g. intercalating with the target nucleic acid, wherein as a consequence of the interaction, the expression, i.e. transcription and/or translation, of said target nucleic acid is inhibited or downregulated, preferably inhibited. A regulator directed against a target nucleic acid may also be a molecule, which enhances the cleavage or degradation of this nucleic acid.

The term “P2Y₆ polypeptide” refers to the P2Y purinoceptor 6, while the term “P2Y₆ nucleic acid” refers to a nucleic acid such as DNA or mRNA encoding the P2Y purinoceptor 6.

A compound or regulator according to the invention may be selected from:
(i) nucleic acids, in particular small interfering RNA (siRNA), micro RNA (miRNA) or a precursor thereof, oligonucleotide aptamers, anti-sense oligonucleotides, or ribozymes;
(ii) peptidic compounds, in particular antibodies or antibody fragments or peptidic aptamers;
(iii) small organic non-peptidic molecules, i.e. molecules having a low molecular weight; and
(iv) combinations thereof.

Such compounds or regulators may have the ability to specifically regulate at least one target as described herein, e.g. due to binding to the at least one target.

The term compound as it appears herein refers inter alia to a molecule that is able to change or regulate or modulate the activity of the P2Y₆ polypeptide. This change may be an increase or a decrease in signaling activity, binding characteristics, functional, or any other biological property of the polypeptide. In order to reduce food intake, body weight and/or improve glucose homeostasis, inhibition of the P2Y purinoceptor 6 is advantageous.

In another preferred embodiment, the action of the P2Y purinoceptor 6 is impeded by interference to its nucleic acid, which can be both DNA and RNA, by inactivation, degradation, downregulation, or intercalation. Inactivation of a nucleic acid can happen for instance by methylation of nucleotides, insertion, deletion, nucleotide exchange, cross linkage, or strand break/damage. Downregulation of DNA or RNA is referred to as diminished expression of these nucleic acids and can happen by binding of repressors, which are usually polypeptides, but can also happen by chemical or structural changes or modifications of the nucleic acids. Intercalation is the reversible inclusion of a molecule between two other molecules. In nucleic acids, intercalation occurs when ligands of an appropriate size and chemical nature fit themselves in between base pairs. According to the invention, compounds for the inhibition of P2Y purinoceptor 6 polypeptide can be molecules like small molecules, RNA or DNA molecules, siRNA or precursor thereof, miRNA or precursors thereof, ribozymes, DNA or RNA antisense oligonucleotides, aptamers, antibodies or fragments thereof, peptides, polypeptides, cyclopeptides.

The inventive compounds are also referred to as regulators. They regulate the expression and/or activity of the P2Y purinoceptor 6 polypeptide and can be identified using one or more assays, alone or in combination. Test compounds used in the screening are not particularly limited. They can be either artificial or natural. The term small molecule refers to low molecular weight organic compounds being by definition not a polymer. In the field of pharmacology, it is usually restricted to a molecule that also binds with high affinity to a biopolymer such as proteins, nucleic acids, or polysaccharides. The upper molecular weight limit for a small molecule is approximately 200 Da, which allows for the possibility to rapidly diffuse across cell membranes. Small molecules are broadly used as enzyme inhibitors or receptor blockers, thus they are preferred regulators for the inhibition of P2Y purinoceptor 6 polypeptide in the present invention. Preferred small molecules according to the invention are selected from general formula (I)

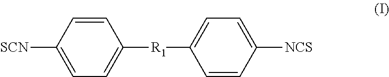

wherein

R₁ is selected from: trans-CH═CH—, —CH₂—CH₂—, —NHCSNH(CH₂)₂NHCSNH—, —NHCSNH(CH₂)₃NHCSNH—, —NHCSNH(CH₂)₄NHCSNH—

or said compound is selected from general formula (II)

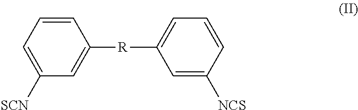

wherein

R is selected from: —NHCSNH(CH₂)₂NHCSNH—, —NHCSNH(CH₂)₃NHCSNH—, —NHCSNH(CH₂)₄NHCSNH—, and salts and solvates thereof.

It is preferred that the compound according to the invention is selected from the group comprising or consisting of 1,4-Di[3-(3-isothiocyanatophenyl)thioureido]butane, 1-isothiocyanato-4-[2-(4-isothiocyanatophenyl)ethyl]benzene, 1-amino-4-[[4-[[4-chloro-6-[(3-sulfophenyl)amino]-1,3,5-triazin-2-yl]amino]-3-sulfophenyl]amino]-9,10-dioxoanthracene-2-sulfonic acid. These compounds are all inhibitors of P2Y₆.

A particular preferred embodiment of the invention refers to the compound 1,4-Di[3-(3-isothiocyanatophenyl)thioureido]butane (also called MRS2578) for use in the treatment of diseases related to energy balance and carbohydrate metabolism and homeostasis.

Said inhibitor of P2Y₆ has a molecular weight of 472.67 and can be described by the following formula:

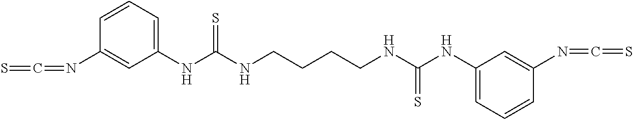 [SMILES: S=C=Nc1cccc(NC(=S)NCCCCNC(=S)Nc2cccc(N=C=S)c2)c1]

1,4-Di[3-(3-isothiocyanatophenyl)thioureido]butane (MRS2578) is a potent P2Y₆ receptor antagonist with IC₅₀ of 37 nM. 1,4-Di[3-(3-isothiocyanatophenyl)thioureido]butane (MRS2578) selectively blocks P2Y₆ receptor activity versus activity at P2Y₁, P2Y₂, P2Y₄ or P2Y₁₁ receptors.

A further preferred embodiment of the invention refers to the compounds described by the following formula:

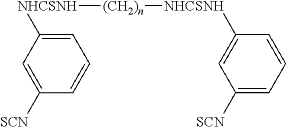

with n=4 or n=3.

Another embodiment of the invention refers to the compounds of formula I or II selected from:

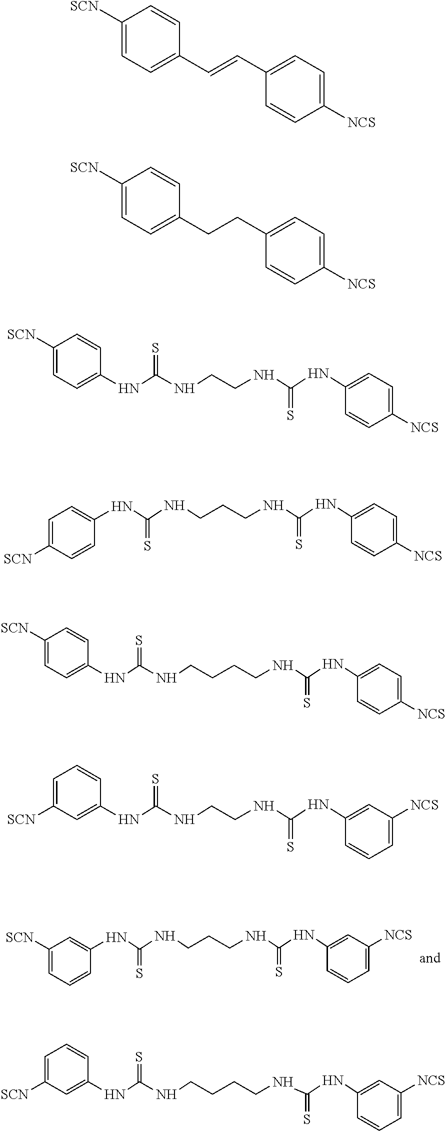

In the context of the present invention, the term “antibody” covers monoclonal antibodies, polyclonal antibodies, multispecific antibodies (e.g. bispecific antibodies) formed from at least two antibodies, antibody fragments and derivates thereof as long as they exhibit the desired activity. The antibody may be an IgM, IgG, e.g. IgG1, IgG2, IgG3 or IgG4. Antibody fragments comprise a portion of an antibody, generally the antigen binding or variable region of the intact antibody. Examples of antibody fragments include Fab, Fab′, F(ab′) 2 and Fv fragments, diabodies, single chain antibody molecules and multispecific antibody fragments. For therapeutic purposes, particularly for the treatment of humans, the administration of chimeric antibodies, humanized antibodies or human antibodies is especially preferred. The antibodies according to the invention may be coupled to a labeling group, particularly for diagnostic applications, e.g. the detection of diseases related to energy balance and carbohydrate metabolism and homeostasis. Examples for suitable labeling groups such as fluorescent groups are known in the art.

A compound acting on protein level may be an aptamer, i.e. an oligonucleic acid, a peptide molecule or a combination thereof that specifically binds to the target polypeptide. Aptamers are mostly short single stranded DNA- or RNA-molecules, which can bind a specific molecule because of their 3D-structure. Aptamers may be prepared by chemical synthesis and selected by a systematic evolution of ligands due to exponential enrichment as known by the person skilled in the art. An oligonucleic acid aptamer may have a sequence length of between about 20-100 nucleotides, preferably about 25-75, more preferably about 30-50 nucleotides. A peptide aptamer generally consists of a variable peptide loop attached at both ends to a protein scaffold. The variable loop length is between 5 and 50, preferably about 10-30, and more preferably about 10-20 amino acids. An aptamer according to the invention may be coupled to a labeling group, particularly for diagnostic applications. Furthermore, particularly for therapeutic applications, the aptamer may be coupled to an effector group, e.g. a cytotoxic group such as toxin.

In a further embodiment, the compound according to the invention is directed against a target nucleic acid, i.e. regulator acting on the nucleic acid level. Preferably, the compound is a nucleic acid compound, e.g. RNAi inducing molecules like siRNA, miRNA, anti-sense oligonucleotide, ribozyme, a precursor or a combination thereof. In a preferred embodiment of the invention, the regulator is an inhibitor of the expression of a target nucleic acid, which preferably acts by down-regulation or knock-down of the target. Down-regulation or knock-down of the target results preferably in a reduction of the steady state level of the target mRNA or polypeptide by at least factor 2, 5, 10 or more, or in a disappearance of the target from the cell.

A RNAi-inducing molecule may refer to a nucleic acid molecule, wherein at least one polynucleotide strand of said nucleic acid molecule has a sequence which is sufficiently complementary to a target RNA, preferably to a target mRNA, in order to effect its processing, i.e. its decomposition. In order to have an RNAi-inducing effect, it is necessary that the complementarity between the RNAi-inducing molecule and a region of the target RNA is sufficient, in order to effect a hybridization and a subsequent processing. For example, the complementarity is at least 80%, preferably at least 90% and most preferably at least 99%, whereby the 5′- and/or 3′-ends as well as the overhangs of an RNAi-effector molecule may also contain nucleotides, which are not complementary to the target RNA.

SiRNA (small interfering RNA or short interfering RNA or silencing RNA) used according to the invention is a double-strand of RNA and/or nucleotide analogues with 3′ overhangs on at least one end, preferably either ends. Each RNA strand of the double-strand has a 5′ phosphate group and a 3′ hydroxyl group. Preferably, each RNA strand of the double strand is 19 to 30 nucleotides long, more preferably 20 to 28 nucleotides and most preferably 21 to 23 nucleotides. The 3′ overhang on the end of a RNA strand is preferably 2 nucleotides long. In a particular preferred embodiment the siRNA double-strand consists of two 21 nucleotides long RNA strands each having a 2 nucleotides long 3′ overhang. SiRNA molecules further refer to single-stranded RNA-molecules having a length of 19-30 nucleotides, preferably 20-28 nucleotides and particularly having a length of 21-23 nucleotides, whereby the single-stranded RNA molecule is for at least 80%, preferably for at least 90% and more preferably for more than 99% complementary to a sequence of a target RNA, in particular of a target mRNA, and a binding of siRNA to the target RNA effects a sequence specific decrease. Preferably, siRNA molecules have overhangs of 1-3 nucleotides on the 3′ end. Methods for obtaining siRNA molecules are known to the person skilled in the art.

MiRNA is a single- or double-stranded RNA molecule of 19-30, preferably 20-28, and more preferably 21-23 nucleotides in length, which can regulate gene expression.

MiRNA is generally synthesized at first as a precursor, which is then processed to the major form having a sequence which is at least partially complementary to messenger RNA of a target molecule according to the invention.

An antisense oligonucleotide may be a single, double, or triple-stranded DNA, RNA, PNA (peptide nucleic acid) or a combination thereof (e.g. hybrids of DNA and RNA strands) having a length of between about 10-100, preferably 20-50, and more preferably 20-30 nucleotides in length, which can interfere with mRNA targets by hybrid formation and therefore inhibit translation of said mRNA.

Ribozymes are catalytic RNAs possessing a well defined structure that enables them to catalyze a chemical reaction. Apart from naturally occurring ribozymes they can be made artificially and be tailored to interact with nucleic acids and proteins. Ribozymes are also preferred modulators for inhibition or activation of the preferred kinases in the present invention.

Precursor molecules, e.g. precursor molecules of siRNA and/or miRNA may be a substrate for the siRNA/miRNA-biogenesis-apparatus of the target cell. This comprises, for example, RNA precursor molecules such as double-stranded RNA (dsRNA) or short hairpin RNA-molecules (shRNA), which are processed by enodribonucleases such as Drosha and/or Pasha to siRNA-molecules or miRNA-molecules, respectively. Dicer is another endoribonuclease that cleaves double-stranded RNA and pre-microRNA (miRNA) into siRNA about 20-25 nucleotides long, usually with a two-base overhang on the 3′ end. Dicer catalyzes the first step in the RNA interference pathway and initiates formation of the RNA-induced silencing complex (RISC). The RISC complex with a bound siRNA recognizes complementray mRNA molecules and degrades them, resulting in substantially decreased levels of protein translation and effectively turning off the gene.

DsRNA-molecules or short hairpin RNA-molecules (shRNA) having a length of more than 27 nucleotides, preferably more than 30 up to 100 nucleotides or longer, and mostly preferred dsRNA-molecules having a length of 30-50 nucleotides, can be used.

Further precursor molecules according to the invention may be DNA constructs encoding dsRNA, shRNA, siRNA and/or miRNA, whereby the coding elements are controlled by regulatory elements allowing an expression of dsRNA, shRNA, siRNA and/or miRNA in the target cell. Examples for such control elements are polymerase II promoters or polymerase III promoters such as, for example, U6 or H1.

SiRNA, miRNA, ribozymes and antisense oligonucleotides may be coupled with a labeling group, e.g. for diagnostic purposes or may be coupled with an effector molecule known in the art and they may be applied to a target cell by any technique which is known to a person skilled in the art, such as transfection of exogenous siRNA, miRNA, ribozyme and antisense oligonucleotide or of an appropriate vector, e.g. viral or non-viral, producing a single transcript which can be processed into a functional siRNA, miRNA, ribozyme or antisense oligonucleotide.

A compound acting on the nucleic acid level as described above may exhibit analogs of one or more nucleotides within its nucleotide sequence. Said nucleotide analogs may, for example, increase the structural stability of the RNA molecule or the stability towards ribonucleases. Ribonucleotide analogs are well known to the person skilled in the art and are modified compared to the original RNA molecules by base modification, sugar modification, e.g. modification of the 2′-OH group of ribose and/or phosphate backbone modifications.

Diseases related to energy balance and carbohydrate metabolism and homeostasis refer to diseases and conditions characterized by pathological disorders of the metabolism. Thereby the term “diseases related to energy balance or energy homeostasis” refers mainly to diseases caused by deregulated amount of energy eaten and/or metabolized. Energy balance is the relationship between energy intake (food calories taken into the body by food and drink) and energy consumption (calories being used in the body). Energy intake refers to the sum of calories consumed as food and energy expenditure is mainly a sum of internal heat produced and external work. Diseases related to energy balance and carbohydrate metabolism and homeostasis are mainly characterized by enzyme defects and abnormalities in the regulating system leading to a pathological enrichment of substrates, lack of metabolic products, failure of producing energy, of regeneration of cellular constituents, of elimination of metabolic products, and of maintenance of homeostasis. They can be acquired or be a genetic disease. Diseases related to energy balance and carbohydrate metabolism and homeostasis as used herein are particularly, but are not limited to, obesity and diabetes. Carbohydrate metabolism denotes the various biochemical processes responsible for the formation, breakdown and interconversion of carbohydrates in living organisms, wherein the most important carbohydrate is glucose. The hormone insulin is the primary regulatory signal in animals; if present, it causes many tissue cells to take up glucose from the circulation, causes some cells to store glucose internally in the form of glycogen, causes some cells to take in and hold lipids, inhibits de novo glucose synthesis in some cells (liver, kidney) and in many cases controls cellular electrolyte balances and amino acid uptake as well. Diseases of the carbohydrate metabolism refer to diseases and conditions characterized in pathophysiological alterations in the metabolism of one or more carbohydrates. It is preferred if the disease of the carbohydrate homeostasis and metabolism is selected of one disease of the group comprising or consisting of obesity, diabetes mellitus, lactose intolerance, fructose intolerance, galactosemia, glycogen storage disease, diabetic ketoacidosis, hyperosmolar coma and hypoglycemia. Particularly preferred within the present invention are obesity, type 2 diabetes and related complications selected from cardiovascular diseases, hepatic steatosis and lipid disorders. Even more preferred are obesity and therapeutic control of insulin resistance or insensitivity in type 2 diabetes.

Another aspect of the present invention refers to a compound for activation of P2Y purinoceptor 6 polypeptide or upregulation or modification for advanced transcriptional activity of a nucleic acid encoding P2Y purinoceptor 6, upregulation of P2Y purinoceptor 6 signaling pathway for the treatment of diseases related to energy balance and carbohydrate metabolism and homeostasis such as for gaining or maintaining weight, anorexia nervosa, cancer cachexia, underweight, especially treatment of underweight of newborns, treatment of underweight of people in extreme need of care. The compounds are suitable for that treatment by increasing appetite.

The body mass index (BMI) is generally used as a means of correlation between groups related by general mass and can serve as a vague means of estimating adiposity. The duality of the BMI is that, while it is easy to use as a general calculation, it is limited as to how accurate and pertinent the data obtained from it can be. Generally, the index is suitable for recognizing trends within sedentary or overweight individuals because there is a smaller margin of error BMI provides a simple numeric measure of a person's thickness or thinness, allowing health professionals to discuss weight problems more objectively with their patients. The current value recommendations are as follow: a BMI from 18.5 up to 25 may indicate optimal weight, a BMI lower than 18.5 suggests the person is underweight, a number from 25 up to 30 may indicate the person is overweight, and a number from 30 upwards suggests the person is obese. Accordingly, people with a BMI of equal to or less than 18.5, preferably equal to or less than 17, more preferably equal to or less than 16 qualify for being underweight. There are a wide variety of contexts where the BMI of an individual can be used as a simple method to assess how much the recorded body weight departs from what is healthy or desirable for a person of that height.

With other words, people in extreme need of care with a BMI of less than 18.5, or generally speaking people with a BMI of less than 18.5, preferably equal to or less than 17, more preferably equal to or less than 16 may be treated according to the present invention.

In another embodiment of the present invention the compound is a small molecule, an RNA molecule, an siRNA molecule, an miRNA molecule, or a precursor thereof, a polypeptide or a ribozyme.

In a further embodiment of the present invention, the compound for the treatment of diseases related to energy balance and carbohydrate metabolism and homeostasis such as for gaining or maintaining weight, anorexia nervosa, cancer cachexia, underweight, treatment of underweight of newborns, treatment of underweight of people in extreme need of care is a small molecule, selected from the general formula (III)

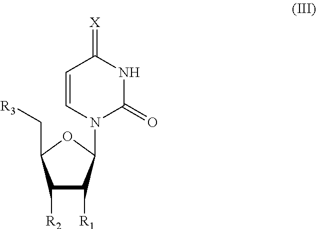

or salts and solvates thereof, wherein X represents ═O or ═S, R₁ represents —H, —OH, —OCHO, —OCOCH₃, —OCOC₂H₅, —OCOC₃H₇, —OCO-cyclo-C₃H₅, —OCOCH(CH₃)₂, —OCOC(CH₃)₃, —OCOC₄H₉, —OCOC₅H₁₁, —OCOCH(CH₃)—C₃H₇, —OCO—CH(CH₃)—C₂H₅, —OCOCH(CH₃)—CH(CH₃)₂, —OCOC(CH₃)₂—C₂H₅, —OCOCH₂—C(CH₃)₃, —OCO—C(CH₃)₃, —OCOCH(C₂H₅)₂, or —OCOC₂H₄—CH(CH₃)₂, and R₂ and R₃ represent independently of each other —OH, —OCHO, —OCOCH₃, —OCOC₂H₅, —OCOC₃H₇, —OCO-cyclo-C₃H₅, —OCOCH(CH₃)₂, —OCOC(CH₃)₃, —OCOC₄H₉, —OCOC₅H₁₁, —OCOCH(CH₃)—C₃H₇, —OCO—CH(CH₃)—C₂H₅, —OCOCH(CH₃)—CH(CH₃)₂, —OCOC(CH₃)₂—C₂H₅, —OCOCH₂—C(CH₃)₃, —OCO—C(CH₃)₃, —OCOCH(C₂H₅)₂, or —OCOC₂H₄—CH(CH₃)₂.

It is particular preferred that said small molecule of general formula (III) represents uridine preferably with (S) conformation of the ribose moiety, triacetyluridine or 4-thiouridine, even more preferably wherein the compound of general formula (III) represents uridine preferably with (S) conformation of the ribose moiety.

In another embodiment of the present invention, the compound for the treatment of diseases related to energy balance and carbohydrate metabolism and homeostasis such as for gaining or maintaining weight, anorexia nervosa, cancer cachexia, underweight, especially treatment of newborns being underweight, treatment of people in extreme need of care being underweight is a small molecule, selected from the general formula (IV)

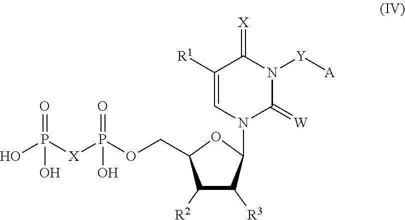

or a salt thereof,

wherein:

A is

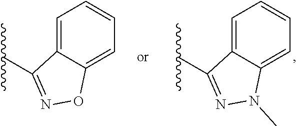 [SMILES: *c1noc2ccccc12 ; *c1nn(C)c2ccccc12]

and wherein A is optionally further substituted with one or more R⁷;

X is selected from —O—, —S—, —N(R⁵)—, —CH₂—, —C₂H₄—, —C₃H₆—, —C(CH₃)₂—, and is independently and optionally substituted with one or more R⁴;

Y is a bond or —CH₂, —C₂H₄, —C₃H₆—, —C(CH₃)₂—, —C₄H₈—, —CH₂—C(CH₃)₂—, —CH(CH₃)—C₂H₄—, —C₅H₁₀, —CH(CH₃)—C₃H₆—, —CH₂—CH(CH₃)—C₂H₄—, —CH(CH₃)—C(CH₃)₂—, —C(CH₃)₂—C₂H₄—, —CH(C₂H₅)—, —C₂H₄—CH(CH₃)—, and is independently and optionally substituted with one or more R⁴;

Z and W are each independently selected from ═O, ═S, ═N(R⁵), and ═N—OR⁵;

R¹ is selected from:

—H, halogen, —OR⁵, —CN, —CF₃, —OCF₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃, and is optionally substituted with one or more R⁷;

R² and R³ are independently of each other selected from —OR⁵, —SR⁵, —NR⁵R⁶ and —OC(O)R⁵;

each occurrence of R⁴ is independently selected from:

halogen, —OR⁵, —NO₂, —CN, —CF₃, —OCF₃, —R⁵, 1,2-methylenedioxy, 1,2-ethylenedioxy, —N(R⁵)₂, —SR⁵, —SOR⁵, —SO₂R⁵, —SO₂N(R⁵)₂, —SO₃R⁵, —C(O)R⁵, —C(O)C(O)R⁵, —C(O)CH₂C(O)R⁵, —C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)₀₋₂NHC(O)R⁵, —N(R⁵)N(R⁵)COR⁵, —N(R⁵)N(R⁵)C(O)OR⁵, —N(R⁵)N(R⁵)CON(R⁵)₂, —N(R⁵)SO₂R⁵, —N(R⁵)SO₂N(R⁵)₂, —N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, —OP(O)(OR⁵)₂, —P(O)(R⁵)₂, —P(O)(OR⁵)₂, or —P(O)(H)OR⁵);

each occurrence of R⁵ is independently selected from:

—H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃,

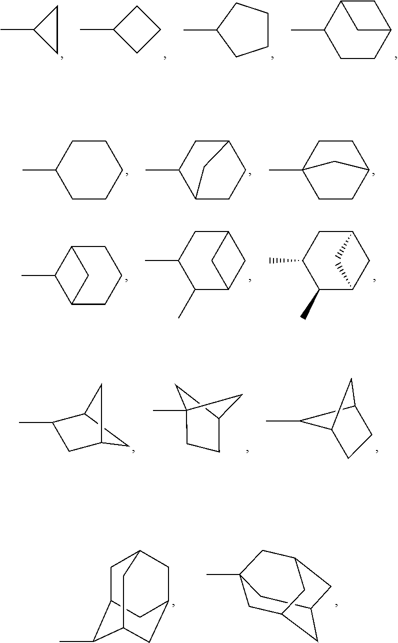

2-thienyl, 3-thienyl, 2-furanyl, 3-furanyl, 2-oxazolyl, 3-oxazolyl, 4-oxazolyl, 2-thiazolyl, 3-thiazolyl, 4-thiazolyl, 1-pyrazolyl, 3-pyrazolyl, 4-pyrazolyl, 5-pyrazolyl, 1-imidazolyl, 2-imidazolyl, 4-imidazolyl, 5-imidazolyl, phenyl, 1-naphthyl, 2-naphthyl, 2-pyridyl, 3-pyridyl, 4-pyridyl, 2-pyrimidinyl, 4-pyrimidinyl, 5-pyrimidinyl, 2-pyrazinyl, 3-pyridazinyl, 4-pyridazinyl, 1,3,5-triazin-2-yl,

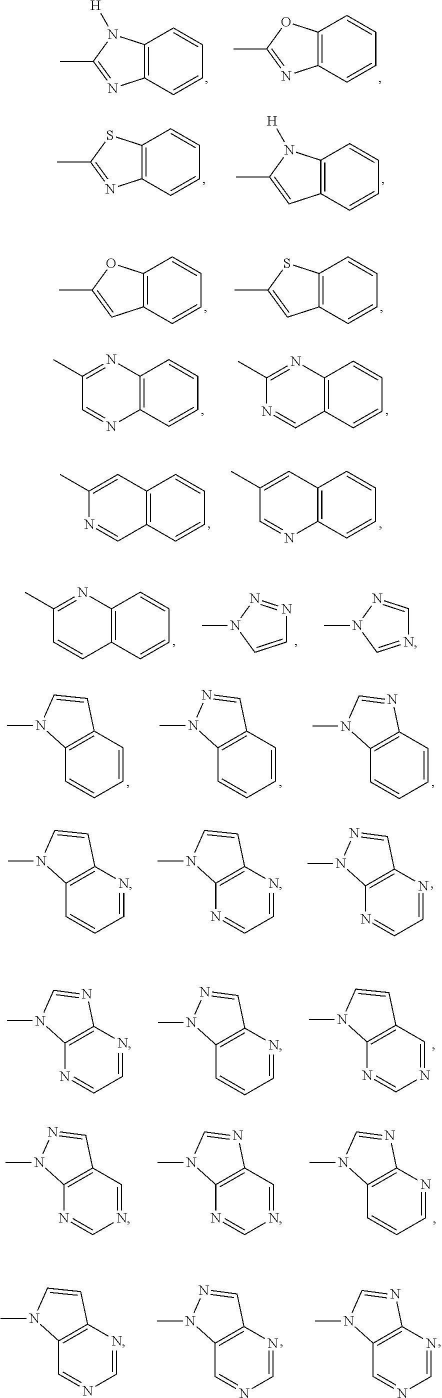

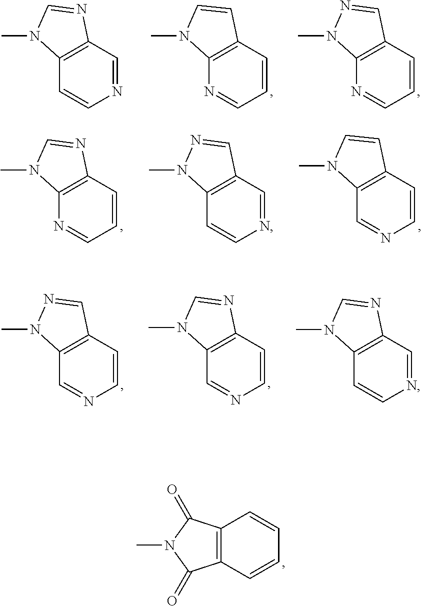

wherein two R⁵ groups bound to the same atom optionally form a 3- to 6-membered aromatic or non-aromatic ring having up to 3 heteroatoms independently selected from N, O, S, SO, or SO₂, wherein said ring is optionally fused to a (C6-C10)aryl, (C5-C10)heteroaryl, (C3-C10)cycloalkyl, or a (C3-C10)heterocycloyl;

and wherein each R⁵ group is independently and optionally substituted with one or more R⁷;

R⁶ is selected from:

—R⁵, —C(O)R⁵, —C(O)OR⁵, —C(O)N(R⁵)₂ and —S(O)₂R⁵;

each occurrence of R⁷ is independently selected from:

halogen, —OR⁸, —NO₂, —CN, —CF₃, —OCF₃, —R⁸, oxo, thioxo, 1,2-methylenedioxy, 1,2-ethylenedioxy, —N(R⁸)₂, —SR⁸, —SOR⁸, —SO₂R⁸, —SO₂N(R⁸)₂, —SO₃R⁸, —C(O)R⁸, —C(O)C(O)R⁸, —C(O)CH₂C(O)R⁸, —C(S)R⁸, —C(S)OR⁸, —C(O)OR⁸, —C(O)C(O)OR⁸, —C(O)C(O)N(R⁸)₂, —OC(O)R⁸, —C(O)N(R⁸)₂, —OC(O)N(R⁸)₂, —C(S)N(R⁸)₂, —(CH₂)₀₋₂NHC(O)R⁸, —N(R⁸)N(R⁸)COR⁸, —N(R⁸)N(R⁸)C(O)OR⁸, —N(R⁸)N(R⁸)CON(R⁸)₂, —N(R⁸)SO₂R⁸, —N(R⁸)SO₂N(R⁸)₂, —N(R⁸)C(O)OR⁸, —N(R⁸)C(O)R⁸, —N(R⁸)C(S)R⁸, —N(R⁸)C(O)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, —OP(O)(OR⁸)₂, —P(O)(R⁸)₂, —P(O)(OR⁸)₂, or —P(O)(H)(OR⁸); and

each occurrence of R⁸ is independently selected from:

—H—CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃.

It is further preferred that said small molecule of general formula (IV) represents:

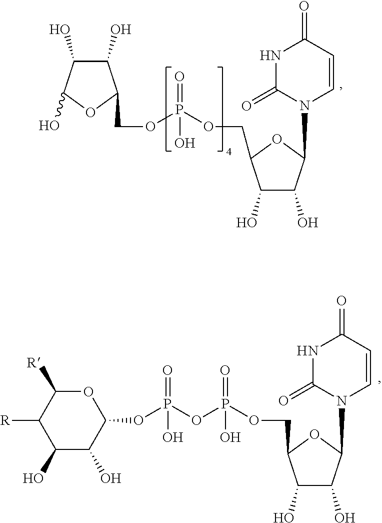

wherein R═OH and R′═COOH, or CH₂OH,

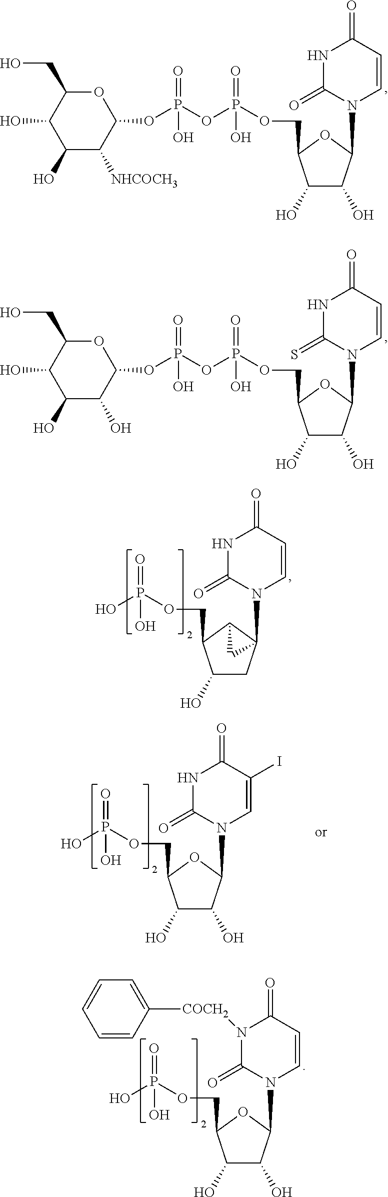

It is further preferred that said small molecule is selected from the group consisting of:

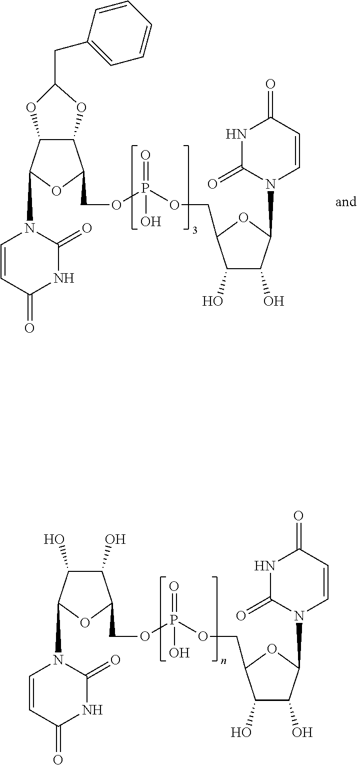

wherein n=3.

Uridine is attractive for therapeutic use because of its low toxicity. Major limiting factors in increasing uridine doses are fever and diarrhea. Oral administration of uridine can increase uridine level in plasma as well as in brain.

In another embodiment of the present invention, the compound is a uridine precursor, uridine, uridine derivative, UDP or an activator of UDP synthesis, any regulator to increase UDP half-life or an activator of P2Y₆ pathway for use in a therapy for increasing food intake and fat mass.

In yet another embodiment of the present invention the compound is uridine wherein the ribose moiety is preferably in the (S) conformation or uridine diphosphate (UDP) derivatives, which are derived from a similar compound by some chemical or physical process. However, the ribose moiety of the uridine, UDP or UDP-derivative is preferably in the (S) conformation. Furthermore, uridine, UDP, or UDP-derivatives are preferably for use in a therapy increasing food intake and fat mass. In yet another embodiment of the present invention the compound is an activator of P2Y₆ pathways or a compound that increases the synthesis of UDP, preferably for use in a therapy increasing food intake and fat mass.

The term “uridine precursor” as used herein refers to any chemical compound preceding uridine in the uridine biosynthesis or may be converted into uridine when administered. The term “uridine derivative” as used herein refers to any compound that is derived from uridine by some chemical or physical process and can bind to P2Y purinoceptor 6 or activate the P2Y₆ pathways.

Uridine is the natural precursor of UDP in the CNS. Thus, when administering uridine, the level of UDP in the hypothalamus is increased. UDP directly activates orexigenic NPY/AgRP neurons via activation of P2Y₆, which is in perfect accordance with its ability to promote feeding.

Increase of food intake is suitable for patients if they have lost a lot of weight due to illness, which may be because of problems with reduced appetite or impaired food resorption. Thus, it is preferred that uridine is used (administered) for therapies increasing food intake in the treatment of cancer cachexia, treatment of anorexia nervosa, treatment of underweight, especially treatment of newborns being underweight, and treatment of people in extreme need of care being underweight.

Enhancement of food intake is also desirable in animal breeding. Therefore another aspect of the present invention refers to uridine for use as stimulant of animal weight. The present invention refers also to the use of uridine precursor, uridine derivative, an activator of UDP synthesis, any regulator to increase UDP half-life or an activator of P2Y₆ pathway in animal feed, preferably as stimulant of food intake.

Furthermore, the present invention provides a method for stimulating increased rate of growth, greater amount of growth and greater feed efficiency in domesticated animals, said method comprising:
a) activation of P2Y purinoceptor 6 polypeptide or
b) upregulation or modification for advanced transcriptional activity of a nucleic acid encoding P2Y purinoceptor 6,
c) upregulation of P2Y purinoceptor 6 signaling pathway
d) activation of uridine transport across the blood brain barrier,
e) activation or increase of UDP synthesis in the CNS,
f) prevention of UDP degradation

The term “domesticated animals” refers in particular to cattle, pigs, sheep and other fattening animals.

The invention relates generally to veterinary pharmaceutical compositions and formulations that
a) stimulate P2Y purinoceptor 6 polypeptide or
b) upregulate a nucleic acid encoding P2Y purinoceptor 6,
c) prevent degradation of a nucleic acid encoding P2Y purinoceptor 6,
d) activation of uridine transport across the blood brain barrier,
e) activation or increase of UDP synthesis in the CNS,
f) prevention of UDP degradation
g) upregulate P2Y purinoceptor 6 signaling pathway.

Another embodiment of the present invention relates to compounds that increase UDP synthesis and thereby stimulate animal food intake.

The present invention concerns a method of stimulating increased rate of growth, greater amount of growth and greater feed efficiency in food and fattening animals which comprises providing to such animals biodegradable and non-biodegradable compressed tablets loaded with
a) an activator for P2Y purinoceptor 6 polypeptide or
b) an activator for upregulation of a nucleic acid encoding P2Y purinoceptor 6,
c) an inhibitor of degradation of a nucleic acid encoding P2Y purinoceptor 6,
d) an activator for uridine transport across the blood brain barrier,
e) an activator for or enhancer for UDP synthesis in the CNS,
f) an inhibitor for UDP degradation
g) an activator for upregulation of P2Y purinoceptor 6 signaling pathway.

The method of the present invention provides advantages over methods known in the art such as, inter alia, increased weight gain.

For use as a medicament, the inventive compound may be formulated as a pharmaceutical composition. Therefore still another aspect of the present invention deals with pharmaceutical compositions comprising at least one compound as defined according to the invention as an active ingredient. The medication comprising a compound as described according to the invention can be formulated to be suitable for any known dosage form. The composition may be administered by one dose per day or may be divided up to several doses. The effective amount of the active agent, i.e. the compound according to the invention, in the composition may be determined by the skilled person without undue burden depending on the kind of active agent and the kind of conditions to be treated. For example, about 1 μg/kg to 15 mg/kg of a regulator may be administered to a human patient, e.g. by one or more separate administrations or by continuous infusion. A typical daily dosage may range from about 1 μg/kg to about 100 mg/kg or more, depending on the factors such as age, gender and weight of the person to be treated etc.

A pharmaceutical composition as defined in the present invention may be further administered as a part of a combination therapy or combinatorial therapy. In the context of the present invention, “combination therapy” or “combinatorial therapy” also refers to the simultaneous administration of two or more active agents, wherein at least one of these agents is a compound according to the present invention. The two or more active agents may be administered simultaneously in one single pharmaceutical composition or more than one pharmaceutical composition, wherein each composition comprises at least one active agent.

The invention relates also to pharmaceutical compositions comprising or consisting of at least one compound according to the invention for the treatment of a disease of the energy balance and carbohydrate metabolism and homeostasis. In another embodiment the pharmaceutical compositions comprises an effective amount of at least one inventive compound, and at least one pharmaceutically acceptable carrier, excipient, binders, disintegrates, glidents, diluents, lubricants, coloring agents, sweetening agents, flavoring agents, preservatives, solvent or the like. The pharmaceutical compositions of the present invention can be prepared in a conventional solid or liquid carrier or diluents and a conventional pharmaceutically-made adjuvant at suitable dosage level in a known way.

According to the invention, the inventive compound or the pharmaceutical composition can be used for the treatment of diseases related to energy balance and carbohydrate metabolism and homeostasis to modulate or regulate food intake, body weight and glucose levels.

The inventive pharmaceutical composition is formulated to be compatible with its intended route of administration. Administration forms include, for example, pills, tablets, film tablets, coated tablets, capsules, liposomal formulations, micro- and nano-formulations, powders and deposits. Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain the compound according to the present invention. Intravenous, intramuscular and oral applications are preferred forms of administration in the present invention, wherein oral application is particularly preferred.

The present invention also includes artificial mammalian milk as well as mammalian milk substitutes as a formulation for oral administration of the inventive compound to newborns, toddlers, infants either as pharmaceutical preparations, and/or as dietary food supplements.

The inventive compound can also be administered in form of its pharmaceutically active salts. Suitable pharmaceutically active salts comprise acid addition salts and alkali or earth alkali salts. For instance, sodium, potassium, lithium, magnesium or calcium salts can be obtained.

The pharmaceutical compositions according to the present invention will typically be administered together with suitable carrier materials selected with respect to the intended form of administration, i.e. for oral administration in the form of tablets, capsules (either solid filled, semi-solid filled or liquid filled), powders for constitution, aerosol preparations consistent with conventional pharmaceutical practices. Other suitable formulations are gels, elixirs, dispersible granules, syrups, suspensions, creams, lotions, solutions, emulsions, suspensions, dispersions, and the like. Suitable dosage forms for sustained release include tablets having layers of varying disintegration rates or controlled release polymeric matrices impregnated with the active components and shaped in tablet form or capsules containing such impregnated or encapsulated porous polymeric matrices. The pharmaceutical compositions may be comprised of 5 to 95% by weight of the inventive compound.

As pharmaceutically acceptable carrier, excipient and/or diluents can be used lactose, starch, sucrose, cellulose, magnesium stearate, dicalcium phosphate, calcium sulfate, talc, mannitol, ethyl alcohol (liquid filled capsules).

Suitable binders include starch, gelatine, natural sugars, corn sweeteners, natural and synthetic gums such as acacia, sodium alginate, carboxymethyl-cellulose, polyethylene glycol and waxes. Among the lubricants that may be mentioned for use in these dosage forms, boric acid, sodium benzoate, sodium acetate, sodium chloride, and the like. Disintegrants include starch, methylcellulose, guar gum and the like. Sweetening and flavouring agents and preservatives may also be included where appropriate. Some of the terms noted above, namely disintegrants, diluents, lubricants, binders and the like, are discussed in more detail below.

Additionally, the compounds or regulators of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations being intended to be converted to liquid form preparations for either oral or parenteral administration, shortly before use. Such liquid forms include solutions, suspensions and emulsions.

The inventive compound may also be delivered transdermally. The transdermal compositions may take the form of creams, lotions, aerosols and/or emulsions and can be included in a transdermal patch of the matrix or reservoir type as are conventional in the art for this purpose.

The term capsule refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art. Oral gels refer to the active ingredients dispersed or solubilized in a hydrophilic semi-solid matrix.

Powders for constitution refer to powder blends containing the active ingredients and suitable diluents which can be suspended in water or juices. One example for such an oral administration form for newborns, toddlers and/or infants is a human breast milk substitute which is produced from milk powder and milk whey powder, optionally and partially substituted with lactose.

Human breast milk is a complex fluid, rich in nutrients and in non-nutritional bioactive components. It contains all of the nutrients needed by the newborn baby. These include the metabolic components (fat, protein, and carbohydrates), water, and the raw materials for tissue growth and development, such as fatty acids, amino acids, minerals, vitamins, and trace elements.

The compounds of the present invention may be components of artificial mother milk formulations in order to increase food intake of newborns having underweight. Artificial mother milk formulations or mother milk substitutes of the present invention are preferably prepared by adding to a mother milk formulation including commercially available mother milk formulations especially in powder form of the compound of the present invention. The inventive compound is preferably added in an amount of 3-100 μg compound or per 100 ml (commercially available) mother milk formulation, more preferably in an amount of 5-70 μg/100 ml and most preferably in an amount of 10-40 μg/100 ml mother milk formulation.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluents in the composition can range from about 5 to about 95% by weight of the total composition, preferably from about 25 to about 75%, more preferably from about 30 to about 60% by weight, and most preferably from about 40 to 50% by weight.

The term disintegrants refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 1 to about 40% by weight of the composition, preferably 2 to about 30% by weight of the composition, more preferably from about 3 to 20% by weight of the composition, and most preferably from about 5 to about 10% by weight.

Binders characterize substances that bind or “glue” powders together and make them cohesive by forming granules, thus serving as the “adhesive” in the formulation. Binders add cohesive strength already available in the diluents or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropyl-methylcellulose; polyvinylpyrrolidone; and inorganics such as magnesium aluminum silicate. The amount of binder in the composition can range from about 1 to 30% by weight of the composition, preferably from about 2 to about 20% by weight of the composition, more preferably from about 3 to about 10% by weight, even more preferably from about 3 to about 6% by weight.

Lubricant refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate and polyethylene glycols. Lubricants are usually added at the very last step before compression, since they must be present on the surfaces of the granules and in between them and the parts of the tablet press. The amount of lubricant in the composition can range from about 0.05 to about 15% by weight of the composition, preferably 0.2 to about 5% by weight of the composition, more preferably from about 0.3 to about 3%, and most preferably from about 0.3 to about 1.5% by weight of the composition.

Glidents are materials that prevent caking and improve the flow characteristics of granulations, so that flow is smooth and uniform. Suitable glidents include silicon dioxide and talc. The amount of glident in the composition can range from about 0.01 to 10% by weight of the composition, preferably 0.1% to about 7% by weight of the total composition, more preferably from about 0.2 to 5% by weight, and most preferably from about 0.5 to about 2% by weight.

Coloring agents are excipients that provide coloration to the composition or the dosage form. Such excipients can include food grade dyes and food grade dyes adsorbed onto a suitable adsorbent such as clay or aluminum oxide. The amount of the coloring agent can vary from about 0.01 to 10% by weight of the composition, preferably from about 0.05 to 6% by weight, more preferably from about 0.1 to about 4% by weight of the composition, and most preferably from about 0.1 to about 1%.

Liquid form preparations include solutions, suspensions and emulsions. Water or water-propylene glycol solutions for parenteral injections may be mentioned as an example. Liquid form preparations may also include solutions for intranasal administration.

Techniques for the formulation and administration of the compound of the present invention may be found in “Remington's Pharmaceutical Sciences” Mack Publishing Co., Easton Pa. A suitable composition comprising the compound mentioned herein may be a solution of the compound in a suitable liquid pharmaceutical carrier or any other formulation such as tablets, pills, film tablets, coated tablets, dragees, capsules, powders and deposits, gels, syrups, slurries, suspensions, emulsions, and the like.

Still another aspect of the present invention relates to the use of the inventive compound as a dietary supplement. That dietary supplement is preferably for oral administration and especially but not limited to administration to newborns, toddlers, and/or infants. A dietary supplement is intended to supplement the diet. The “dietary ingredients” in these products may in addition include: vitamins, minerals, herbs or other botanicals, amino acids, and substances such as enzymes, organ tissues, glandulars, and metabolites.

Dietary supplements may be manufactured in forms such as tablets, capsules, softgels, liquids, or powders.

The invention refers further to a method for screening for a compound for treatment of diseases related carbohydrate metabolism and homeostasis, wherein the method comprises:
a) providing a test compound for contacting at least one P2Y₆ polypeptide
b) detecting the binding of said test compound to the P2Y₆ polypeptide, and
c) determining the activity of the P2Y₆ polypeptide in the presence of said test compound.

The invention further relates to a method for screening for a compound for treatment of diseases related to energy balance and carbohydrate metabolism and homeostasis, the method comprising
a) contacting a test compound with a P2Y purinoceptor 6 polypeptide,
b) detecting the binding of said test compound to the P2Y purinoceptor 6 polypeptide, and
c) determining the activity of the P2Y purinoceptor 6 polypeptide in the presence of said test compound,

wherein instead of polypeptides nucleic acids encoding the polypeptides are used and the expression rate is determined instead of the activity, preferably wherein the test compound is RNA or a peptide or an antibody or a small molecule.

It is preferred that the inventive methods refer to screening for a compound for use in the treatment of obesity or type 2 diabetes as diseases related to energy homeostasis, carbohydrate metabolism and homeostasis.

The term “contacting” as used herein refers to the step wherein the test compound dissolved in water or a mixture of water and a water-soluble organic solvent such as DMSO, acetone, ethanol, methanol, tetrahydrofuran, DMF, ethylacetate, and isopropanol, is mixed with a P2Y purinoceptor 6 polypeptide in water so that the test compound could bind to the P2Y purinoceptor 6 polypeptide in case the test compound has any affinity to the P2Y purinoceptor 6 polypeptide.

The screening method of the present invention apparently consists of three steps. The term test compound may be any of the potential compounds listed above. The contacting of the test compound with at least one P2Y purinoceptor 6 polypeptide can happen e.g. in the form of a compound library, in physiological or non-physiological solution, or solid phase systems, however a liquid environment is preferred. The conditions and the time need to be sufficient to allow the test compound to bind to the polypeptide. The method is normally carried out in solution at room temperature and at a suitable pH value normally between pH 5 and 9, all parameters being easily selected by a skilled person. The P2Y₆ polypeptide can be obtained by purification from primary human cells, cell lines or from cells being transfected with expression constructs which contain the nucleic acid sequences encoding a P2Y₆ polypeptide.

In a preferred screening method of the present invention the test compound is not contacted with a P2Y purinoceptor 6 polypeptide but with nucleic acids encoding the polypeptides and the expression rate in regard to these nucleic acids is determined instead of the polypeptide activity. It is preferred that the test compound is RNA or a peptide or an antibody or a small molecule.

The nucleic acid sequences encoding a P2Y₆ polypeptide can be obtained by cloning the relevant gene, amplification of the cDNAs or chemical synthesis of the nucleic sequences. For the expression of the corresponding polypeptides the nucleic acid sequences can be inserted into expression vectors, such as recombinant bacteriophage, plasmid, or cosmid DNA expression vectors.

The term binding refers to an interaction between the test compound and one or more a P2Y₆ polypeptide or the nucleic acids encoding a P2Y₆ polypeptide. For binding to a protein, the binding interaction is dependent upon the presence of a particular structure of the test compound, e.g. a binding site of an antagonist or an agonist recognized by the receptor. For binding of compounds to nucleic acids, test compounds need to have a complementary sequence to the nucleic acids, or fit into certain secondary or tertiary structures of the nucleic acids.

The binding of the test compounds to the polypeptide or nucleic acids can be checked by any convenient method known in the art. A separation step may be included to separate bound from unbound components. To check whether the test compound has been bound by the polypeptide or nucleic acid, it is advantageous if the test compound and/or the P2Y₆ polypeptide is labeled for direct detection (radioactivity, luminescence, fluorescence, optical or electron density etc.) or indirect detection (e.g., epitope tag such as the FLAG, V5 or myc epitopes, an enzyme tag such as horseradish peroxidase or luciferase, a transcription product, etc.). The label may be bound to a substrate, to the proteins employed in the assays, or to the test compound being the candidate pharmacological agent. The binding of a test compound can also be conveniently checked if one of the components is immobilized on a solid substrate.

Protein-DNA interactions can be for instance checked by gel shift or band shift assays or electrophoretic mobility shift assays (EMSA), which is based on the observation that complexes of protein and DNA migrate through a non-denaturing polyacrylamide gel more slowly than free DNA fragments.

The interactions between peptides or proteins, respectively, can be investigated by various methods, which include, but are not limited to, protein binding microarray, antibody microarrays, protein chips, a variety of assays and UV-crosslink experiments.

In all methods to identify compounds that regulate (activate or inhibit) the expression and signaling activity (via the corresponding G-protein) of the P2Y₆, the expression level and signaling activity are compared to those detected in the absence of the test compound. The present invention is related particularly to the identification of compounds, which have regulatory activity on the signaling activity of the P2Y₆ receptor. Consequently, it is particularly the inhibition or activation of expression and signaling activity that is measured.

The inhibition or activation of nucleic acids on the mRNA-level encoding the P2Y₆ polypeptides can be checked by investigating the expression of the polypeptides by quantitative methods, e.g. Western blot or enzyme-linked immune-adsorbent assay (ELISA). A way to quantify the protein expression is further the measuring of fusion proteins, wherein the polypeptides of the invention are fused to proteins or protein fragments, which are easy to quantify, like fluorescent proteins. The inhibition or activation of DNA and thus the production of mRNA can be checked by mRNA-quantification. Levels of mRNA can be quantitatively measured by Northern blotting. Another way is the reverse transcription quantitative polymerase chain reaction (RT-PCR followed by qPCR).

The inhibition or activation of the polypeptides on the protein-level can be investigated by measuring their activity. The determination of the activity of a polypeptide/protein/enzyme depends on its specificity. Consequently, the activity of G-protein coupled receptors is measured in assays, wherein a substrate of the signaling pathway downstream of the receptor and its G-protein is quantified such as cAMP. Measuring the radioactivity of phosphate being part of cAMP is one possibility.

P2Y₆ polypeptides are also useful in competition binding assays in methods designed to discover compounds that interact with the receptor (e.g. binding partners and/or ligands).

Thus, a compound is exposed to a P2Y₆ polypeptide under conditions that allow the compound to bind or to otherwise interact with the polypeptide. A known ligand such as UDP is also added to the mixture. If the test compound interacts with the receptor, it competes with UDP for the binding to the receptor. Thus, the amount of bound/unbound UDP is changed in presence of the test compound compared to the absence of the test compound. This type of assay is particularly useful in cases to investigate if compounds bind to the receptor at its UDP-binding site.

The invention is further related to a method for treatment of diseases related to energy balance and carbohydrate metabolism or homeostasis, preferably obesity, diabetes mellitus type 2 and associated diseases comprising:
administering a subject in need thereof a therapeutically effective amount of at least one compound for:
a) inhibition of P2Y purinoceptor 6 polypeptide,
b) inactivation, degradation, downregulation or intercalation of a nucleic acid encoding P2Y purinoceptor 6 or
c) downregulation of P2Y purinoceptor 6 signaling pathway.

The invention is further related to a method for treatment of diseases related to energy balance and carbohydrate metabolism or homeostasis, preferably obesity, diabetes mellitus type 2 and associated diseases comprising:
administering a subject in need thereof a therapeutically effective amount of at least one compound for:
a) inhibition of UDP synthesis,
b) inhibition of UDP synthesis in the CNS,
c) inhibition of uridine transport to the CNS,
d) inhibition of uridine transport across the blood brain barrier,
e) acceleration or increase of UDP degradation,
f) acceleration or increase of UDP degradation in the CNS, or
g) inhibition of enzymes important for the synthesis of UDP in the CNS.

In regard to said method, the term “a subject in need thereof” refers in regard to said method to a patient having the risk to develop diseases related to energy balance and carbohydrate metabolism or homeostasis, in particular to develop obesity or a diabetes type 2 or it refers to a patient that has already developed and, thus, suffers from obesity or diabetes mellitus type 2. The term “a therapeutically effective amount” refers to an amount of a compound sufficient to at least reduce in-vivo food intake and symptoms of diseases related to energy balance and carbohydrate metabolism or homeostasis such as obesity, diabetes type 2 and associated complications.

Additionally the present invention is related to a method of treating diseases related to energy balance and carbohydrate metabolism and homeostasis, preferably obesity or diabetes mellitus type 2 comprising administering to a patient in need of such treatment an effective amount of a compound for
a) inhibition of P2Y purinoceptor 6 polypeptide,
b) inactivation, degradation, downregulation or intercalation of a nucleic acid encoding P2Y purinoceptor 6, or
c) downregulation of P2Y purinoceptor 6 signaling pathway.

Furthermore, the present invention is related to a method of optimizing therapeutic efficacy for treatment of diseases related to energy balance and carbohydrate metabolism and homeostasis, preferably obesity or diabetes mellitus type 2, comprising:
(a) Administering an inhibitor of P2Y purinoceptor 6 polypeptide to a subject having said diseases related to energy balance and carbohydrate metabolism and homeostasis; or
(b) Inactivating, degrading, down regulating or intercalating a nucleic acid encoding P2Y purinoceptor 6 in said subject having said diseases related to energy balance and carbohydrate metabolism and homeostasis.

Further the present application relates to a compound for
(i) inhibition of P2Y purinoceptor 6 polypeptide,
(ii) inactivation, degradation, downregulation or intercalation of a nucleic acid encoding P2Y purinoceptor 6 or
(iii) downregulation of P2Y purinoceptor 6 signaling pathway
for the manufacture of a medicament for the treatment of a disease related to energy balance or carbohydrate metabolism and homeostasis such as obesity, type 2 diabetes and associated complications.

Preferably, a compound or regulator according to the invention is used in medicine and more preferred for the treatment of a disease related to energy balance and carbohydrate metabolism and homeostasis such as obesity and diabetes mellitus type 2.

A compound known to affect and in particular to decrease the expression and/or activity of the polypeptides of P2Y₆ purinoceptor can be used for the treatment of diseases related to energy balance and carbohydrate metabolism and homeostasis, preferably obesity, diabetes mellitus, more preferably diabetes mellitus type 2, and preferably for obesity by administration of said compound(s) within pharmaceutical compositions as outlined above.

The compounds or compositions according to the invention are useful for each single disease of the group comprising or consisting of diseases related to energy balance and carbohydrate metabolism and homeostasis, preferably obesity and diabetes type 2.

The invention is further related to a method for treatment of a disease being related to energy balance and carbohydrate metabolism and homeostasis such as cancer cachexia, underweight, especially treatment of newborns being underweight, treatment of people in extreme need of care being underweight comprising:
administering a subject in need thereof a therapeutically effective amount of at least one compound for:
a) activation of P2Y purinoceptor 6 polypeptide,
b) activation, upregulation of a nucleic acid encoding P2Y purinoceptor 6,
c) upregulation of P2Y purinoceptor 6 signaling pathway,
d) activation of Uridine transport across the blood brain barrier,
e) activation or increase of UDP synthesis in the CNS,
f) prevention of UDP degradation, or
g) prevention of UDP degradation in the CNS.

The invention is further related to a method for treatment of a disease being related to energy balance and carbohydrate metabolism or homeostasis such as cancer cachexia, underweight, treatment of newborns being underweight, treatment of people in extreme need of care being underweight comprising:
administering a subject in need thereof a therapeutically effective amount of at least one compound for:
a) activation of UDP synthesis,
b) activation of uridine transport to the CNS, or
c) activation of enzymes important for the synthesis of UDP in the CNS.

The term “a subject in need thereof” refers in regard to said method to a patient with cancer cachexia, underweight, or a newborn with underweight, or people in extreme need of care with underweight. The term “a therapeutically effective amount” refers in regard to said method to an amount of a compound sufficient to at least increase in-vivo food intake and fat mass.

Preferably, a compound or regulator described herein is used in medicine and more preferred for the treatment of a disease related to energy balance and carbohydrate metabolism and homeostasis such as cancer cachexia, underweight, treatment of newborns being underweight, treatment of people in extreme need of care being underweight.

A compound known to affect and in particular to increase the expression and/or activity of the polypeptides of P2Y₆ purinoceptor can be used for the treatment of cancer cachexia, underweight, treatment of underweight by newborns, treatment of underweight by people in extreme need of care by administration of the inventive compound(s) within pharmaceutical compositions as outline above.

The compounds or compositions according to the invention are useful for each single disease of the group comprising or consisting of cancer cachexia, underweight, preferably underweight of newborns, underweight of people in extreme need of care.

One embodiment of the invention relates to a method for the diagnosis of diabetes type 2, comprising: providing a sample, preferably a blood or serum sample, from a subject suspected of having diabetes type 2; and detecting the level of uridine in the sample.

The uridine level measured is compared to standardized uridine level or to previous values of the respective patient. Thereby elevated uridine level is indicative for diabetes type 2.

TABLE 1
Sequence identities of the target genes and target proteins

| | | NCBI Accession |
|---|---|---|
| SeqID No. | Sequence Name | PRI 15 Feb. 2014 |
| 1 | P2Y₆ cDNA | NM_001277204 |
| | | Version: NP_001264133.1 |
| 2 | P2Y₆ protein | NP_001264133 |
| | | Version: NP_001264133.1 |
| 3 | P2RY₆ gene | NC_000011.10 |
| | | Chr 11:_72.98-73.01 Mb |

The embodiments in the description and the following examples are provided by way of illustration of the invention and are not included for the purpose of limiting the invention. The variations and changes of the invention which are obvious to a person skilled in the field and solutions equivalent to embodiments described herein fall within the scope of protection of the patent claims.

### EXAMPLES

The inventors found that hypothalamic UDP levels are abnormally regulated upon obesity and diabetes. Indeed, hypothalamic UDP levels are increased in nutritionally as well as genetically-induced obese/diabetic mice (respectively the diet-induced obese mice fed a high-fat diet (HFD) as compare to normal chow diet-fed (NCD) mice and the genetically obese/diabetic mice) (FIG. 7C-7D). The increased-UDP levels associated with obesity and diabetes seems to be driven by increased circulating uridine levels, as plasma uridine levels are increased in obese/diabetic mice and hypothalamic UDP levels positively correlates with circulating uridine (FIG. 8A, 8C). Interestingly, the inventors found that this increased circulating levels of uridine is also present in human diagnosed with T2D, suggesting the existence of convergent uridine/UDP regulation across species and therefore, that this work is also relevant in man (FIG. 8B).

The inventors could show that elevated circulating uridine are associated with diabetes in human as serum uridine levels are increase in patients diagnosed with type 2 diabetes (FIG. 1E). That correlation suggests a particular, but not limited to, utilization of uridine level for diagnosis of disease related to energy balance and carbohydrate metabolism and homeostasis, preferably diabetes mellitus type 2.

### Example 1

Investigation of the regulation of P2Y₆ and its ligand uridine diphosphate (UDP) in the hypothalamus of obese and diabetic mice was performed. For that purpose nutritionally as well as genetically-induced obese/diabetic mice were used. Diet-induced obese mice, C57BL/6N males (purchased from Charles River) received high-fat diet (HFD) or control normal chow diet (NCD) starting at 8 weeks of age until sacrifice (20 weeks). HFD (purchased from Sniff Diets) contains (in calories from): 21% carbohydrates, 19% protein, and 60% fat (D12492-1) and NCD (purchased from Sniff Diets) contains 70% carbohydrates, 20% protein, and 10% fat (D12450B). Mice homozygous for the mutated form of the leptin receptor isoform b (db/db mice) and their control littermates were purchased from the Jackson Laboratory. Mice were housed as described above, briefly in individual cages under specific pathogen-free conditions, maintained in a temperature-controlled room and provided ad libitum access to water and, unless stated otherwise, standard laboratory chow-diet. HFD- and NCD-fed mice as well as db/db and control mice were sacrificed by decapitation. Trunk blood was collected for plasma preparation and hypothalami were quickly dissected and frozen. Prior to uridine measurement, mice plasma were deproteinized using perchloric acid treatment (70%; Sigma). Hypothalami were homogenized in 70% methanol solution and lyophilized. Hypothalamic UDP and circulating uridine levels were measured by UPLC. Uridine levels were also measured in patients diagnosed with type 2 diabetes and a control group. Therefore human sera were deproteinized using perchloric acid treatment (70%; Sigma) and circulating uridine levels were measured by UPLC. The control and the type 2 diabetes (T2D) groups were matched regarding sex ratio, age and body weight, but differ from the diagnosis of type 2 diabetes.

It was found that UDP level was increased in the hypothalamus of HFD as well as db/db mice (FIGS. 7C and D), suggesting a critical role of circulating uridine in the regulation of hypothalamic UDP level. Supporting this finding, hypothalamic UDP level positively and significantly correlates with plasmatic uridine (FIG. 8A). In addition, elevated circulating uridine appears to be associated with diabetes in human as serum uridine level increase in patients diagnosed with type 2 diabetes (FIG. 8B).

### Example 2

To explore whether UDP plays a direct role on the central regulation of feeding behavior, the effect of intracerebroventricular administration (ICV) of UDP on spontaneous food intake in wild type mice fed with a normal chow diet was measured. Therefore, mice were anesthetized using ketamine/dexmedetomidine. Guide cannulas (26 gauges) were stereotaxically inserted into the lateral ventricle (bregma −0.2 mm, midline 1.0 mm, dorsal surface −2.1 mm). Animals were allowed to recover for one week prior to experiments.

Prior to the onset of the dark phase, mice acutely received 2 μL ICV administration of UDP (1 μM, 10 μM and 30 μM; Sigma-Aldrich®), MRS 2578 (1 μM, 10 μM; Sigma-Aldrich®), or control solution (NaCl). Spontaneous food intake was measured 2 and 4 hours after ICV administration from pre-weighed portions of food dispensed from the food rack. ICV administration of UDP increases food intake in a dose-dependent manner (FIG. 1A). UDP appears to be a strong orexigenic agent as administration of 30 μM UDP induces a 45% increase of spontaneous food intake.

In an opposite fashion, ICV administration of MRS2578, a non-competitive selective antagonist of P2Y₆ decreases food intake (FIG. 1B). This set of data shows that central UDP and P2Y₆ modulate feeding behavior and that inhibition of P2Y₆ is able to decrease food intake.

### Example 3

To investigate whether P2Y₆ is particularly enriched in the ARH, the inventors next compared the expression levels of P2Y₆-mRNA in different hypothalamic regions such as the ARH, the paraventricular nucleus of the hypothalamus (PVH), the ventromedial nucleus of the hypothalamus (VMH), the dorsomedial nucleus of the hypothalamus (DMH) and the lateral hypothalamic area (LHA). The greatest mRNA-expression level of P2Y₆ was found in the ARH, where it is expressed=30 to 50% higher than in other hypothalamic regions (FIG. 2A). Since P2Y₆ have been described as the bonafide receptor for uridine diphosphate (UDP), the inventors next analyzed whether the expression of P2Y₆ occurs in the same regions where UDP is produced. A critical step in neuronal UDP synthesis relies on phosphorylation of uridine-monophosphate (UMP), which is mediated by uridine-cytidine kinases (UCK)-1 and -2. Expression analyses of UCK-1 and UCK-2 mRNA revealed an overall similar pattern to that one of P2Y₆ (FIGS. 2B and 2C).

### Example 4

Having identified a restricted domain of P2Y₆ expression in neurons of the ARH, the inventors next investigated whether intracerebroventricular (icv) application of the P2Y₆ agonist UDP modulates activation of ARH neurons. To this end, C57BL/6 N control mice were implanted with icy cannulas in the lateral ventricle. Following icy administration of either vehicle (i.e. saline) or 30 μM UDP, brains were processed for immunochemistry with an antibody, which detects the immediate early gene product cFos. Quantitative assessment of cFos immunoreactive cells revealed a 2.5 fold increase in cFos immunoreactivity in the arcuate nucleus of the hypothalamus (FIG. 3A). Since cFos activation can occur either directly in the respective brain area or indirectly via trans-synaptic activation of cells located in projecting areas of directly activated neurons, the inventors investigated the direct effect of UDP on its P2Y₆. Previous studies reported that, in the periphery, UDP action on P2Y₆ lead to tyrosine phosphorylation of MAP kinases ERK-1 and ERK-2 (pERK). Using a hypothalamic cell line, the inventors found that indeed a similar mechanism occurs in immortalized AgRP-neurons as UDP stimulation clearly increased ERK-phosphorylation (FIG. 3C). Therefore, the inventors also used pERK as a direct read-out of UDP action and the inventors assessed pERK immunoreactivity in the arcuate nucleus following icy UDP application. Quantification of pERK immunoreactivity revealed that UDP significantly increased the number of pERK-positive cells in the ARH (FIG. 3B). Taken together, these analyses revealed that P2Y₆ are expressed on ARH neurons and that they are functional as UDP directly activates signaling in these neurons. Given the profound expression of the UDP receptor P2Y₆ in the ARH as well as the clear activation of both cFos-expression and ERK-phosphorylation in the ARH in response to icy UDP application, the inventors next aimed to define the molecular identity of the P2Y₆-expressing, UDP-responsive neurons in this region. Interestingly, UDP-induced pERK immunoreactive cells displayed a specific anatomical distribution where they were mainly located in the ventromedial region of ARH (FIG. 3B).

### Example 5

Given that the ventromedial part of the ARH mainly contains AgRP/NPY neurons, the inventors next directly investigated the specific effect of P2Y₆/UDP on those neurons. The inventors addressed whether icy application of UDP specifically activates NPY/AgRP-coexpressing neurons in the ARH. The inventors therefore repeated the UDP-induced cFos experiments in mice, which express GFP under control of the neuropeptide (NPY)-promotor (NPY-GFP-mice). This analysis revealed, that while in the basal state following vehicle injection only 20% of NPY-neurons exhibited cFos immunoreactivity, this proportion was increased to 60% following icy UDP application (FIG. 4A), indicating that P2Y₆ are not only expressed in orexigenic AgRP/NPY-neurons but also that UDP modulates the activity of these cells in vivo.

Next the inventors aimed to further directly support the notion, that the P2Y₆ agonist UDP can modulate the activity of AgRP-expressing neurons in the ARH. Therefore, the inventors performed electrophysiological recordings from transgenic mice which express red tomato protein under control of the AgRP promoter through Cre-loxP-mediated recombination in (B6; 129S6-Gt(ROSA)26Sortm9(CAG-tdTomato)Hze/J) mice (AgRPtdTomato) (FIGS. 4B and 4C). In accordance with the cFos data in NPY-GFP mice described above, using electrophysiology, the inventors also found that 60% of AgRP-neurons are sensitive to UDP (FIG. 4D). Here, application of 3 μM UDP resulted in increased action potential frequency of AgRP neurons (FIG. 4B, E). Collectively, these experiments indicate that orexigenic AgRP/NPY-coexpressing neurons in the ARH not only express the UDP receptor P2Y₆ but that they are directly activated following UDP application both in vitro and in vivo.

### Example 6

To directly assess, whether the ability of centrally applied UDP to activate feeding depends on AgRP-cell activation, the inventors investigated the effect of centrally administered UDP in mice, which allow for pharmacogenetic inhibition of AgRP cells. To this end, the inventors injected AgRPCre mice bilaterally into the ARH with an AAV, which co-expresses in a Cre-dependent manner the inhibtory DREADD-channel hM4D and mCherry. Immunostanning for m-Cherry revealed successful bilateral targeting of the ARH in these mice (FIG. 5A). While in Cre-negative control animals bilateral injection of the AAV and intraperitoneal CNO-application had no effect on UDP's ability to increase food intake, UDP's food intake stimulatory effect was completely abolished upon CNO-mediated inhibition of AgRP-neurons of AAV-injected AgRPCre mice (FIG. 5B). Taken together, these experiments clearly revealed that centrally applied UDP enhances food intake, and that this effect is abolished, when activation of AgRP-cells is specifically inhibited.

### Example 7

To further elucidate the specific contribution of P2Y₆-mediated signaling in UDP-dependent activation of food intake, the inventors took a pharmacological approach. Thus, the inventors first compared the orexigenic effect of centrally applied UDP either in the absence or presence of the well characterized P2Y₆ antagonist MRS 2578. Again, while UDP injection increased food intake by ≈50% also in these independent set of experiments, this response was completely abrogated upon co-treatment with the P2Y₆ antagonist (FIG. 6A). Importantly, for this experiment the inventors used a dose of MRS 2578 that does not modulate food intake by itself (FIG. 6A). To investigate whether the inhibition of UDP-induced feeding by central administration of MRS 2578 might stem from unspecific side effects of the antagonist, the inventors also tested the effect of MRS 2578 on the ability of centrally applied ghrelin to increase feeding. Here, icy application of 2 μg ghrelin increased feeding to comparable extend as observed upon UDP injection (FIG. 6B). However, co-application of the P2Y₆ antagonist MRS 2578 had no effect on ghrelin's ability to increase food intake (FIG. 6B). These findings clearly provided first evidence that UDP's ability to increase feeding is specifically mediated via P2Y₆-dependent signal transduction.

Having defined that the food intake-promoting activity of UDP is mediated through P2Y₆-dependent signaling, the inventors aimed to assess whether also UDP's ability to activate AgRP-neuron firing depends on functional P2Y₆ signaling. To this end, the inventors performed electrophysiological recordings from genetically identified AgRP-neurons in AgRPtdTomato mice. While application of the P2Y₆ antagonist MRS 2578 alone had no influence on action potential frequency of these cells, MRS 2578-preincubation abrogated UDP's ability to reduce the membrane potential and to activate action potential firing of AgRP-neurons (FIGS. 6C and D). Thus, not only UDP-induced activation of feeding but also UDP-stimulated activation of AgRP-neurons critically depends on functional P2Y₆ signaling.

### Example 8

Having identified a novel regulatory role for UDP-evoked P2Y₆-dependent signaling in control of AgRP-neuron activity and feeding, the inventors assessed whether P2Y₆ expression or UDP concentrations might be altered in the hypothalamus in obesity.

Therefore, the inventors compared the hypothalamic mRNA expression of P2Y₆ in control mice and diet-induced obese animals as well as in control mice and mice carrying a mutation in the leptin receptor gene (db/db-mice). This analysis revealed unaltered expression of P2Y₆ in the hypothalamus of diet-induced and genetically obese animals (FIGS. 7A and B). In contrast, ultra performance liquid chromatography (UPLC)-based assessment of hypothalamic UDP concentrations revealed a significant increase in hypothalamic UDP content in both obese mouse models (FIG. 7C, D). Moreover, hypothalamic UDP concentrations positively correlated both with body weight and markers of impaired glucose homeostasis such as fasting glycemia and HOMA-IR as an indicator or insulin resistance in obese mice (FIG. 7E-F). Collectively, these experiments revealed that under conditions of obesity, hypothalamic concentrations of the P2Y₆ ligand UDP are significantly increased in the absence of alterations in P2Y₆ mRNA-expression.

### Example 9

Since the inventors detected elevated hypothalamic UDP concentrations in both diet-induced and genetically determined obesity, they further investigated potential mechanisms underlying this phenomenon. Thus, the inventors monitored the mRNA-expression of key enzymes of UDP synthesis and conversion. However, there was no consistent alteration in hypothalamic mRNA expression for the key regulatory gene products in UDP synthesis or conversion detectable in the hypothalamus of high-fat diet fed or db/db-mice.

Neuronal UDP synthesis critically depends of the availability of uridine, which reaches the brain via transporter-mediated uptake. Therefore, the inventors compared the expression of known transporters for pyrimidine and pyrimidine metabolites in the hypothalamus of control mice, diet-induced obese animals as well as db/db-mice. Here, two major transporter families, SLC28a and SLC29a have been demonstrated to be of critical importance for uridine and associated metabolites transport across the blood brain barrier. However, there was no significant alteration in the mRNA-expression of SLC28a and SLC29a family members in diet-induced or genetically determined obesity.

In contrast, when the inventors assessed circulating serum uridine concentrations, there was a significant increase in serum uridine concentrations in obese mice (FIG. 8A). Moreover, the inventors detected a striking correlation between serum uridine concentrations and hypothalamic UDP content in these animals (FIG. 8C). Collectively, these data point to the possibility that in obesity serum uridine concentrations increase and subsequently lead to increased hypothalamic UDP synthesis in the absence of alterations in uridine transporter gene expression as well as in the presence of unaltered expression of enzymes required for UDP synthesis.

To directly address whether elevation of serum uridine concentrations—as observed in obesity—can lead to increased hypothalamic UDP synthesis, the inventors next injected C57BL/6 N control animals intraperitoneally with uridine. The acute intraperitoneal injection of 50 mg/kgBW uridine resulted in a rapid increase of serum uridine concentrations 60 minutes post injection and serum levels returned to baseline concentrations 90 minutes after injection (FIG. 9A). This increase in circulating uridine concentrations resulted in subsequent elevation of hypothalamic UDP content as early as 90 minutes after peripheral application of uridine (FIG. 9B). Finally, consistent with the notion that increased hypothalamic UDP concentrations enhance feeding, animals injected intraperitoneally with uridine increased food intake significantly 4 hours after peripheral application of uridine (FIG. 9C). Taken together, the experiments revealed that in obesity circulating uridine concentrations are increased, providing enhanced substrate availability for hypothalamic UDP-synthesis, ultimately promoting feeding via UDP-induced P2Y₆ signaling in the CNS.

All animal procedures were conducted in compliance with protocols approved by local government authorities (Bezirksregierung Kôln; district council Cologne).

Statistics

All values were expressed as the means±SEM. Statistical analyses were conducted using GraphPad PRISM (version 5.0d). Statistical significance was determined using unpaired two-tailed Student's t-tests or one-way analysis of variance (ANOVA) followed by a Bonferroni's poshoc test for experiments with more than two groups. For electrophysiology experiments, data are depicted as boxplots according to Tukey and statistics determined using non-parametric paired student t-test or one-way ANOVA (nonparametric; Friedman-Test). P<0.05 was considered to be statistically significant. *p<0.05, **p<0.01, and ***p<0.001 versus control and treated group.

### DESCRIPTION OF THE FIGURES

FIG. 1: (A) Food intake measurement (depicted as food intake to body weight) after intracereborventricular (icv) administration of increasing doses of UDP (1 μM, 10 μM and 30 μM) or vehicle (saline) (n=26vs13vs11vs14). (B) Food intake measurement (depicted as food intake to body weight) after intracereborventricular (icv) administration of P2Y₆ antagonist MRS2578 (1 μM and 30 μM) or vehicle (saline) (n=9vs5vs5). Data are presented as mean±SEM. **p<0.01, ***p<0.001.

FIG. 2: Quantitative real-time PCR analysis of (A) pyrimidinergic receptor P2Y, G-protein coupled, 6 (P2yr6) (n=6-8 samples per region), (B) uridine-cytidine kinase 1 (Uck1) (n=7-8 samples per region) and (C) uridine-cytidine kinase 2 (Uck2) (n=7-8 samples per region) mRNA expression in key microdissected hypothalamic regions: the paraventricular nucleus of the hypothalamus (PVH), the arcuate nucleus of the hypothalamus (ARH), the ventromedial nucleus of the hypothalamus (VMH), the dorsomedial nucleus of the hypothalamus (DMH) and the lateral hypothalamic area (LHA). Data are presented as mean±SEM.

FIG. 3: Confocal images and quantification comparison of (A) cFos- and (B) pERK-immunoreactive cells in the arcuate nucleus (ARH) of mice after intracerebroventricular administration of vehicle (saline) or 30 μM UDP (n_{cFos}=7vs9; n_{pERK}=5vs6). Scale bar 100 μm. V3, third ventricle. (C) Quantification and representative immunoblots of phosphorylated and total ERK in hypothalamic cell lines after incubation with vehicle (saline) or 1 μM UDP (n=11vs12, from 3 independent experiments). Data are presented as mean±SEM, **p<0.01, ***p<0.001.

FIG. 4: (A) Confocal images and quantitative comparison of ARH cFos immunoreactive cells in NPY-GFP mice after intracerebroventricular administration of vehicle (saline) or 30 μM UDP (n=3 vs. 4). (B) Recording of an AgRPtdTomato neuron showing the increase in action potential frequency in response to the application of 3 μM UDP. The figure displays the rate histogram (upper panel), the original recording (mid panel) and representative original traces with high time resolution (lower panel). (C) Identification of a recorded neuron by antibody staining against tdTomato (red) and a biocytin backfill of the recorded neuron (green). (D) Overall responsiveness to 3 μM UDP of the recorded AgRP neuron population (n=11 in total: 4 non-responsive neurons vs 7 responsive). (E) Quantification of action potential (AP) frequency (n=11). Grey circles mark single recordings responding with a significant increase in AP frequency, open circles are non-responsive neurons. All recordings have been conducted in synaptically isolated neurons. Scale bars: A, 50 μm; B, 100 μm and C, 40 μM and 10 μM in the magnifications, respectively. V3, third ventricle. Data are presented as mean±SEM, *p<0.05 (A) and as boxplots generated according to the “Tukey method” (mean: “+” median: horizontal line) (E). *p<0.05.

FIG. 5: (A) Representative microphotographs of mCherry immunostainng in control and AgRP^{Cre} mice injected bilaterally in the arcuate nucleus (ARH) with Cre-dependent rAAV-hSyn-DIO-hM4D(Gi)-mCherry (mCherry, red; DAPI counterstaining, blue). Scale bar: 100 μm. V3, third ventricle. (B) Food intake measurement in virus-injected AgRP^{Cre} mice or control wild type litter mates. Mice received CNO injections (0.3 mg/kgBW) 15 minutes prior to icy administration of 30 μM UDP or vehicle (Saline) (n=6 vs 8 vs 5 vs 5). Data are presented as mean±SEM, **p<0.01, ***p<0.001 as compare to vehicle.

FIG. 6: Food intake measurement after intracerebroventricular (icv) administration of (A) vehicle (0.1% DMSO), 1 μM of the P2Y6-specific antagonist MRS 2578, μM UDP or co-icv of 30 μM UDP and 1 μM MRS 2578 (n=10vs11vs14vs12), or (B) vehicle (0.1% DMSO), 1 μM MRS 2578, 2 μg ghrelin or co-icv of 2 μg ghrelin and 1 μM MRS 2578 (n=13vs13vs14vs14). (C) Original traces showing the action potential firing during the application of MRS 2578 and during the application of 3 μM UDP in the presence of MRS 2578. (D) Quantification of action potential frequency (AP) (n=12). Data are presented as mean±SEM, *p<0.05 (A-B) and as boxplots generated according to the “Tukey method” (mean: “+”, median: horizontal line) (F). *p<0.05, **p<0.01, ***p<0.001 as compared to vehicle or control.

FIG. 7: Quantitative real-time PCR analysis of pyrimidinergic receptor P2Y, G-protein coupled, 6 (P2yr6) in hypothalamus of (A) diet-induced obese animals fed a high-fat-diet (HFD) or control animals receiving a normal chow diet (NCD) (n=7vs8) and (B) db/db and their control litter mates (n=5vs7). Hypothalamic contents of (C) HFD vs NCD mice (n=10vs9) and (D) in db/db and control mice (n=5vs7). Correlation of hypothalamic UDP and: (E) body weight, (F) fasting glycemia and (G) the homeostatic model assessment index of insulin resistance (HOMA-IR) in NCD and HFD mice (n=18). Data are presented as mean±SEM, **p<0.01, ***p<0.001.

FIG. 8: (A) Serum uridine levels of control and db/db mice (n=9vs10). (B) Serum uridine levels of patients diagnosed with type 2 diabetes and control group (n=160vs238). (C) Correlation of hypothalamic UDP and serum uridine levels (n=29).

FIG. 9: Effects of intraperitoneal injection of uridine (50 mg/kgBW) or vehicle (saline) in C₅₇B16/N mice on (A) serum uridine (n=4-5 per group) and on (B) hypothalamic UDP contents (n=5 per group) 60 and 90 minutes post-injection as well on (C) food intake (n=15 per group). Data are presented as mean±SEM, *p<0.05, **p<0.01, ***p<0.001.

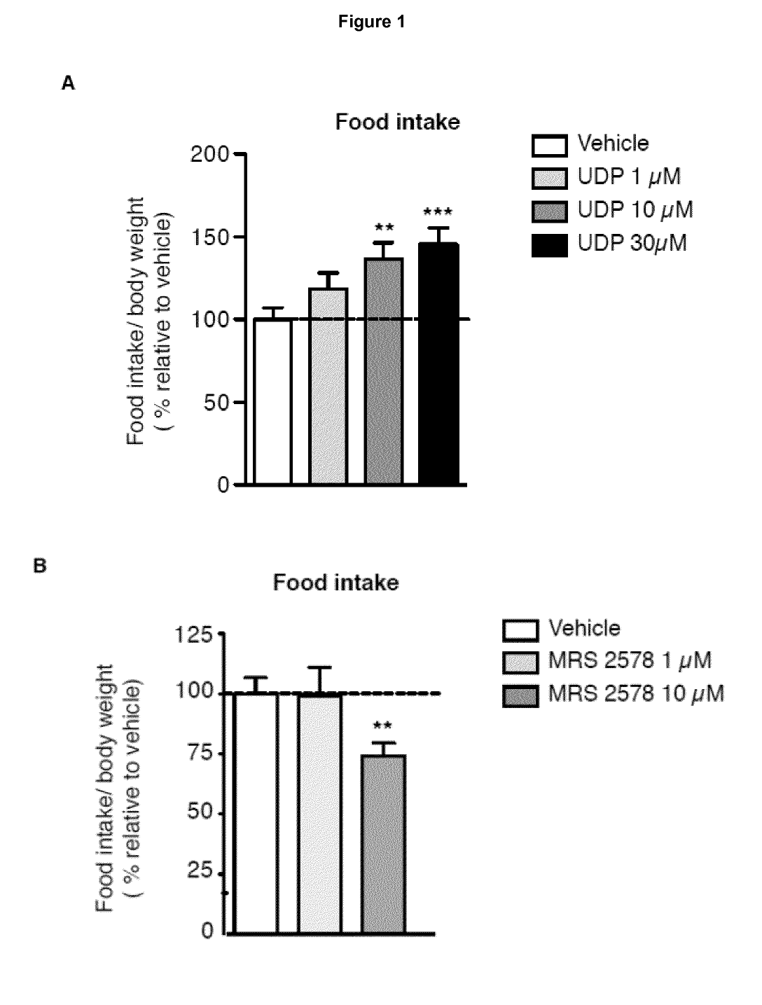

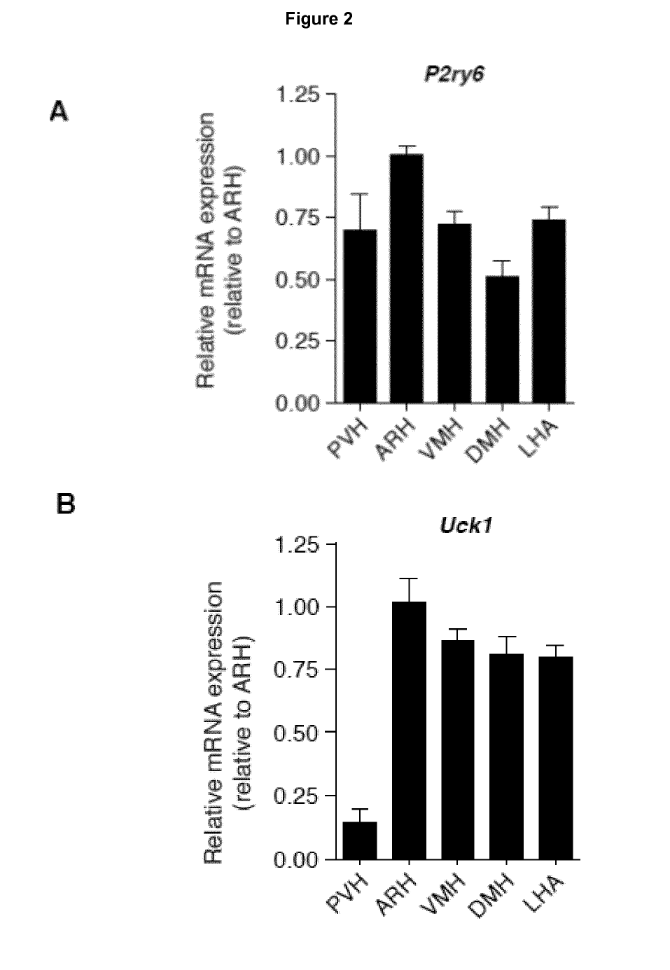

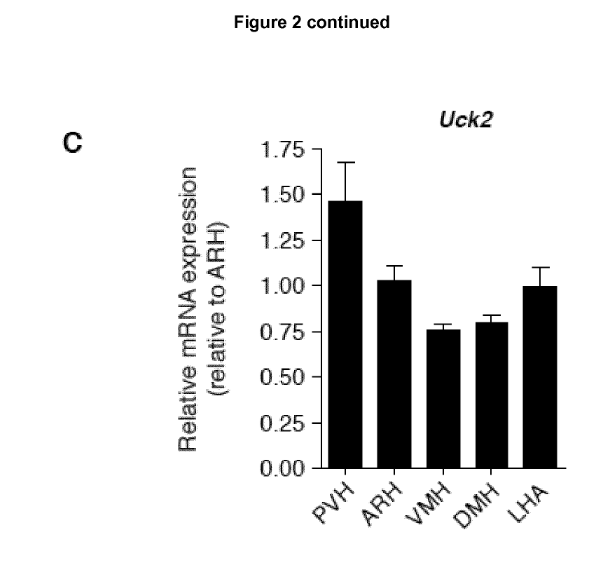

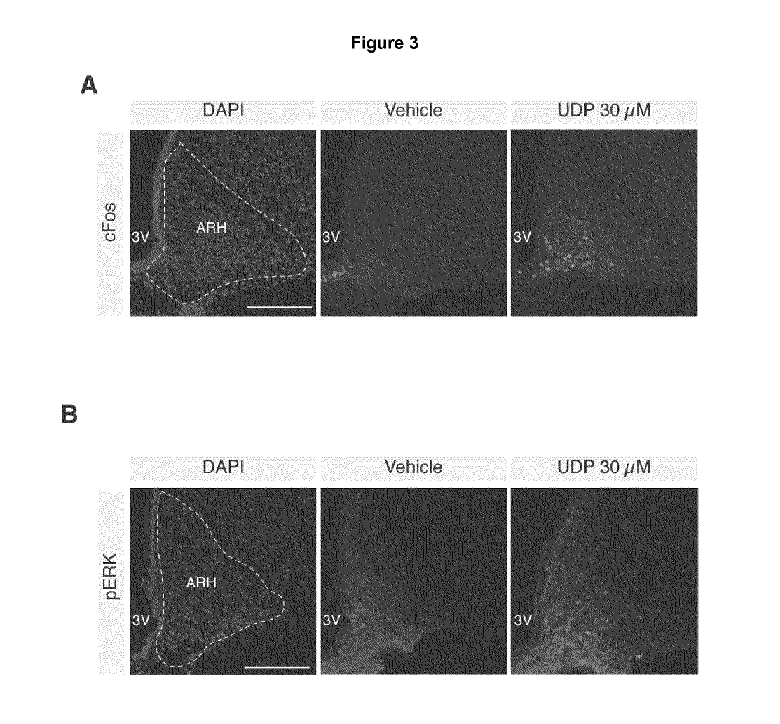

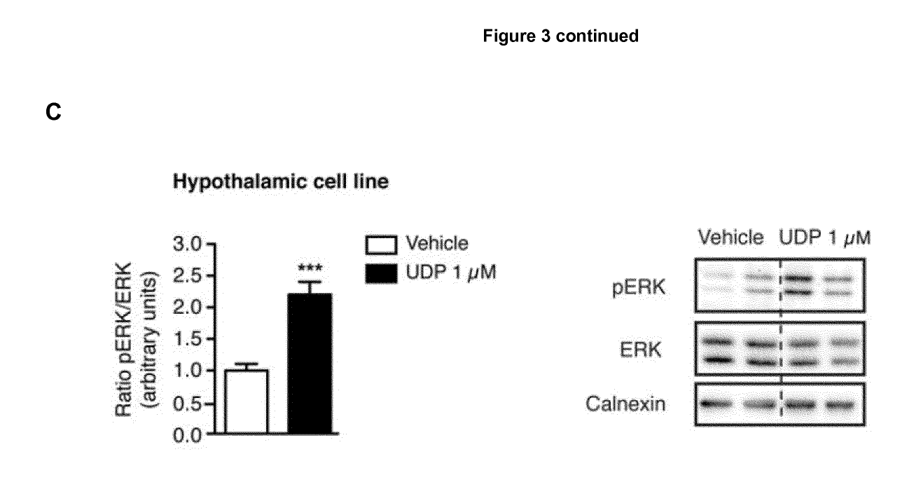

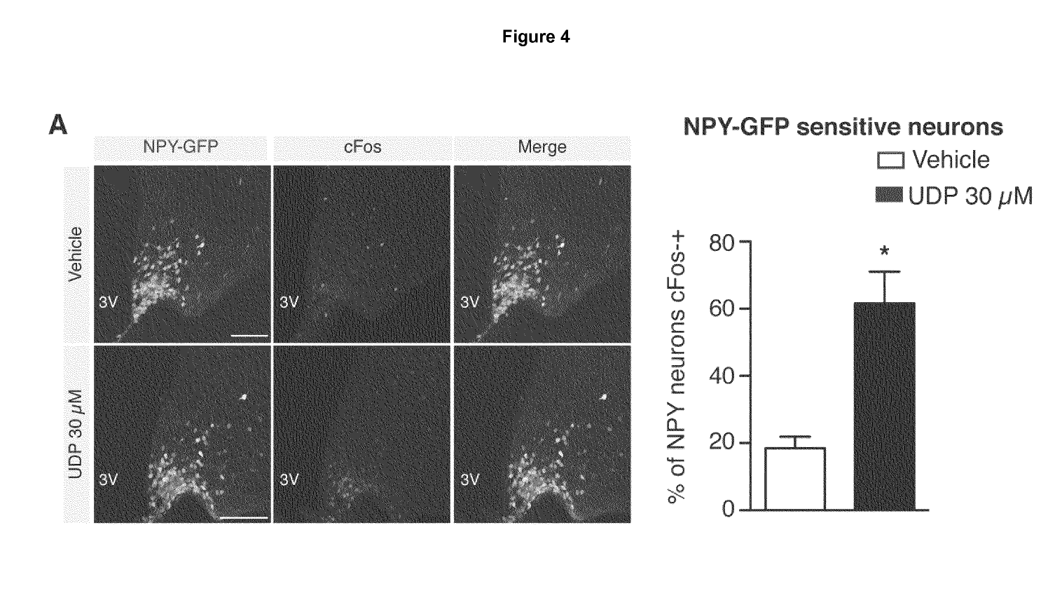

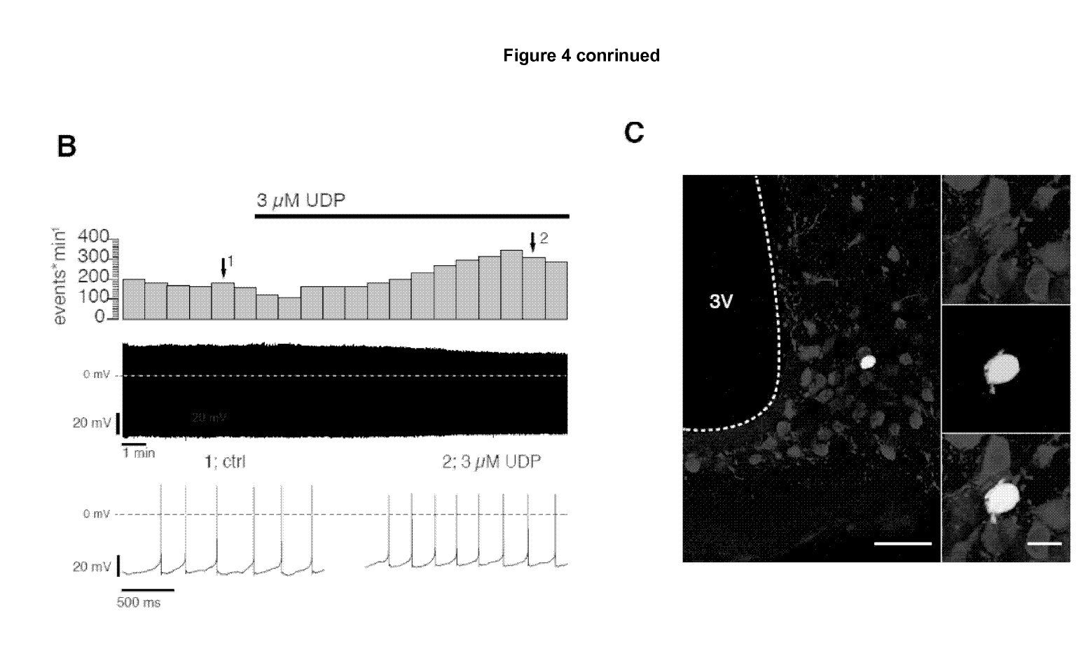

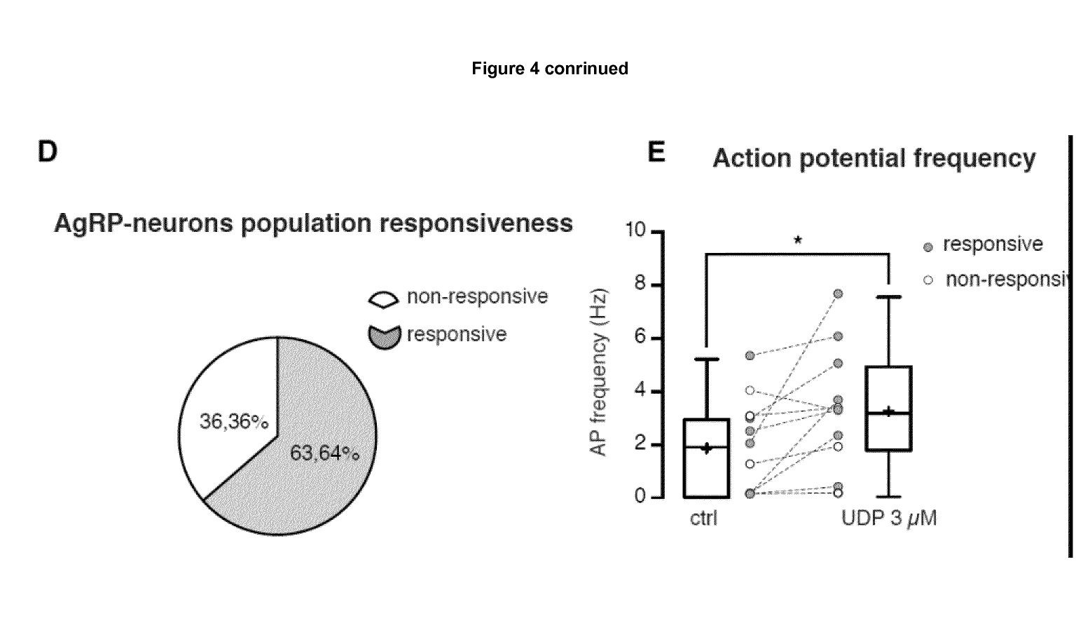

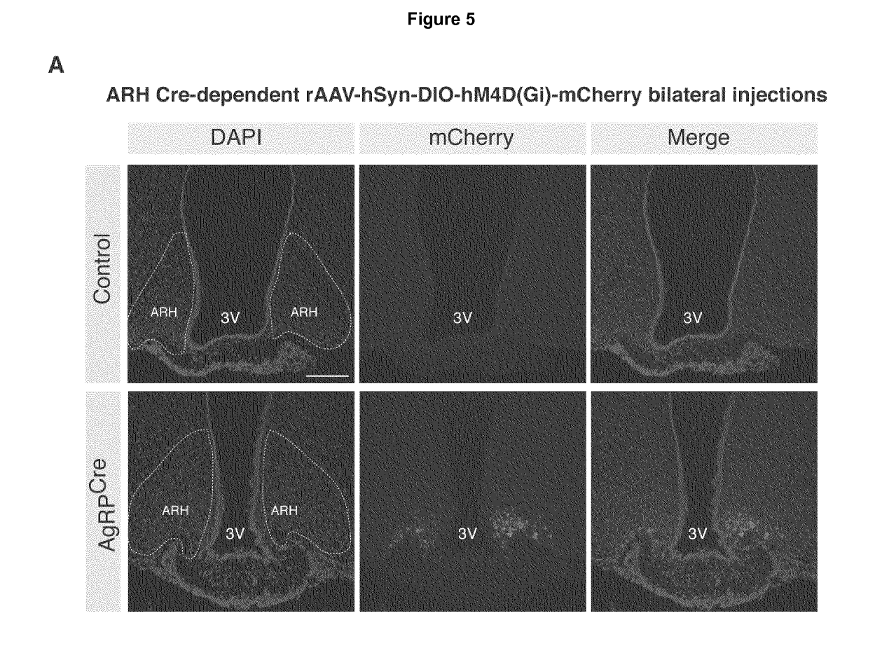

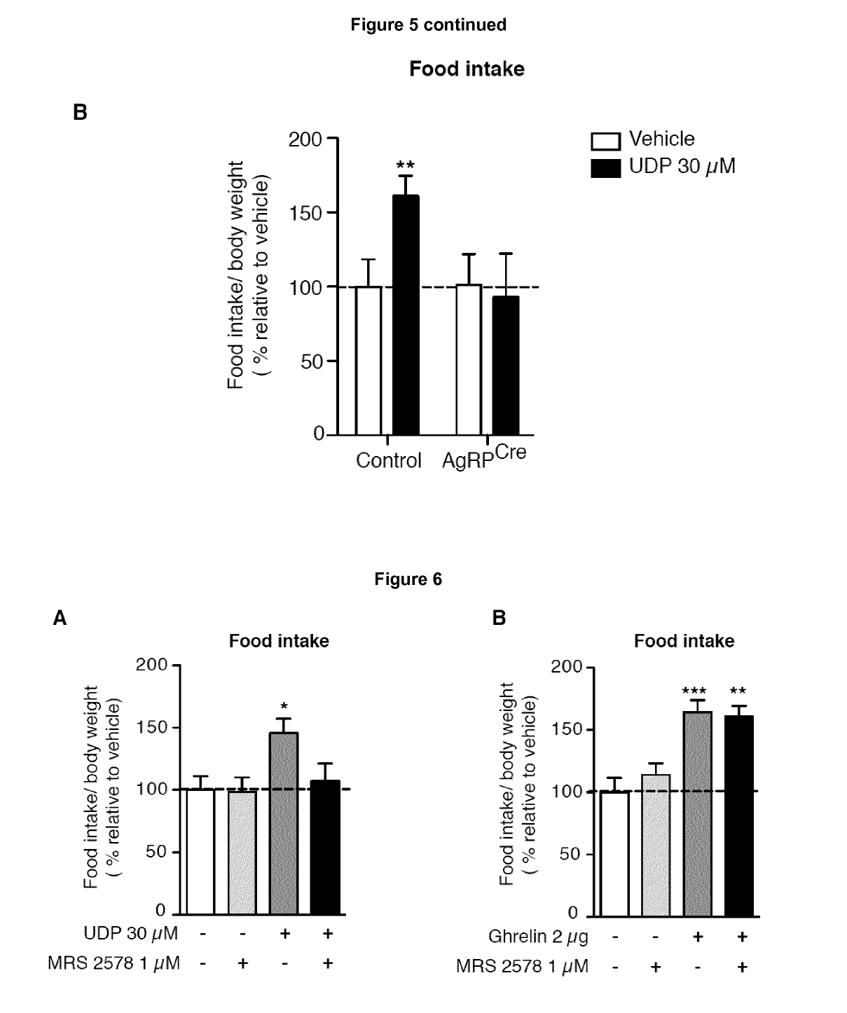

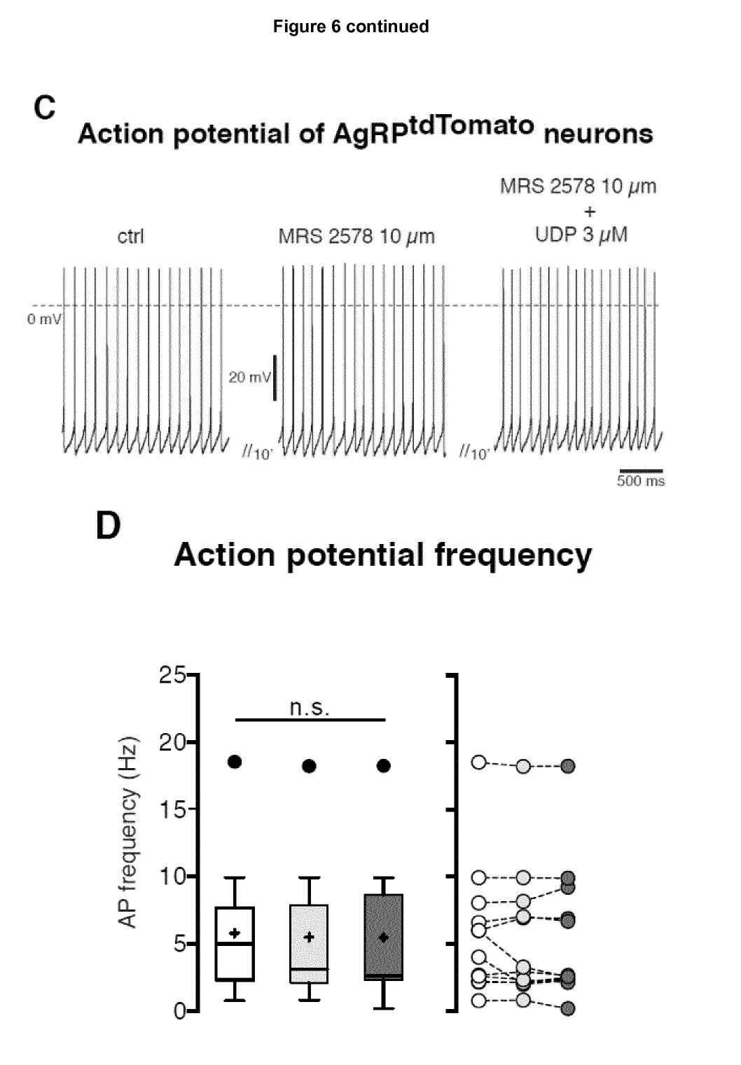

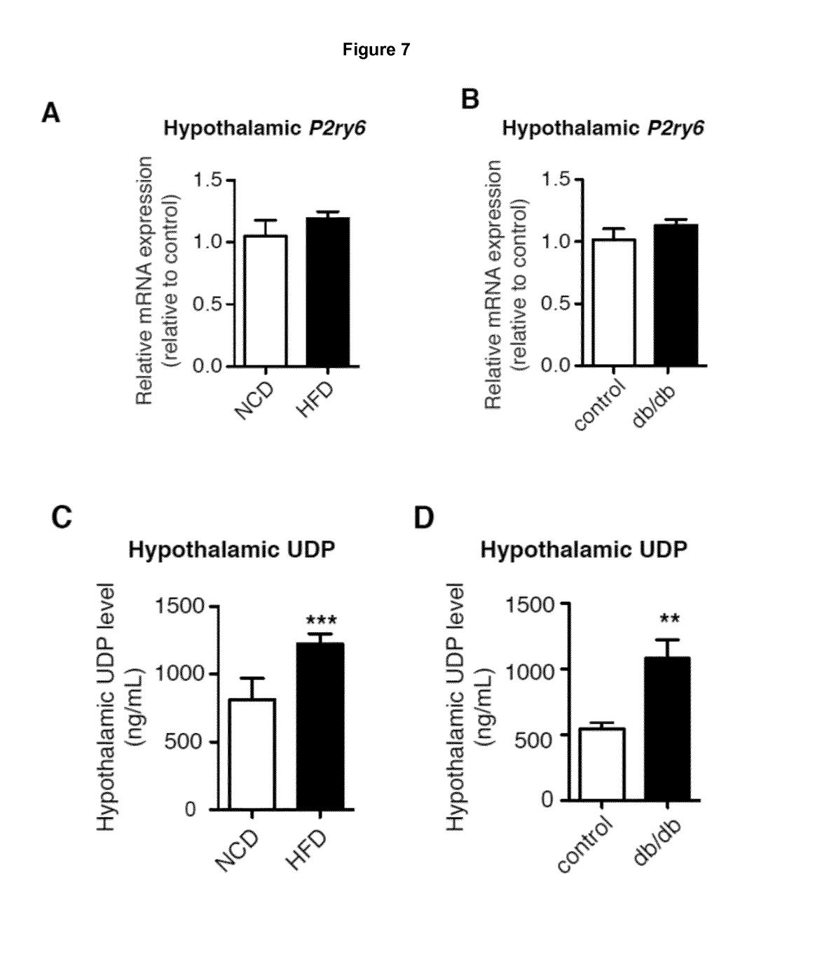

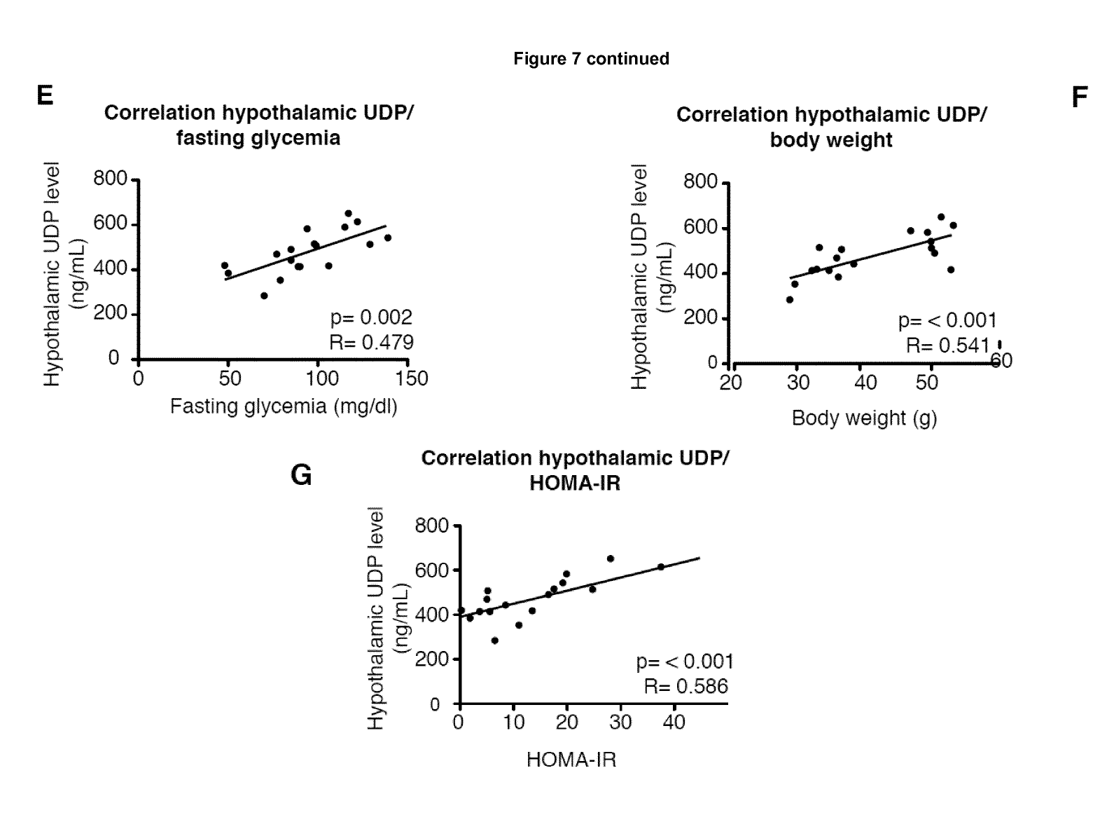

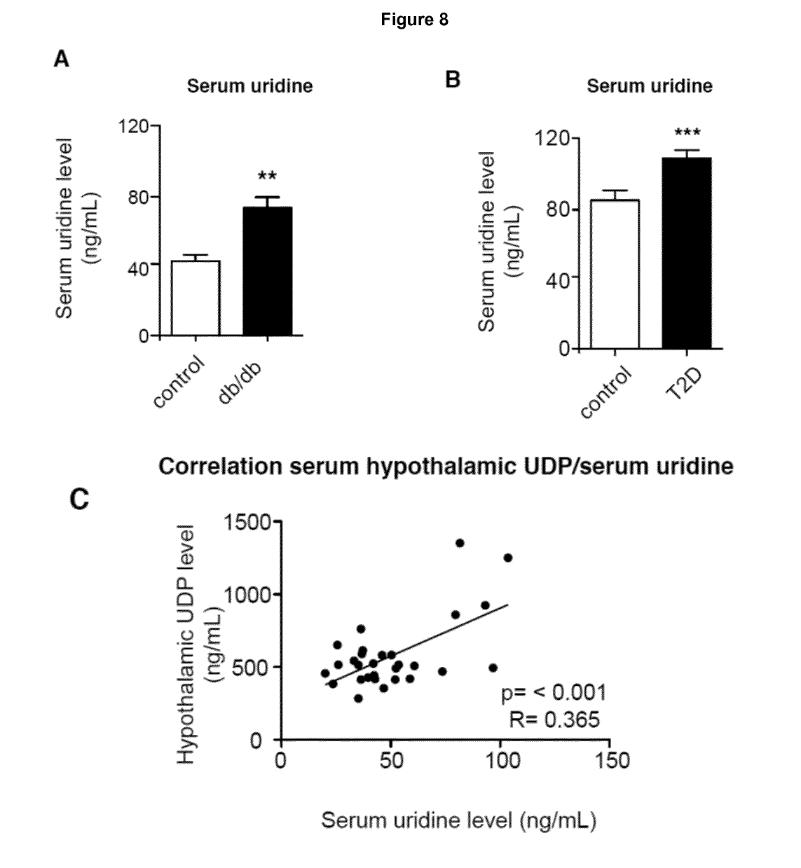

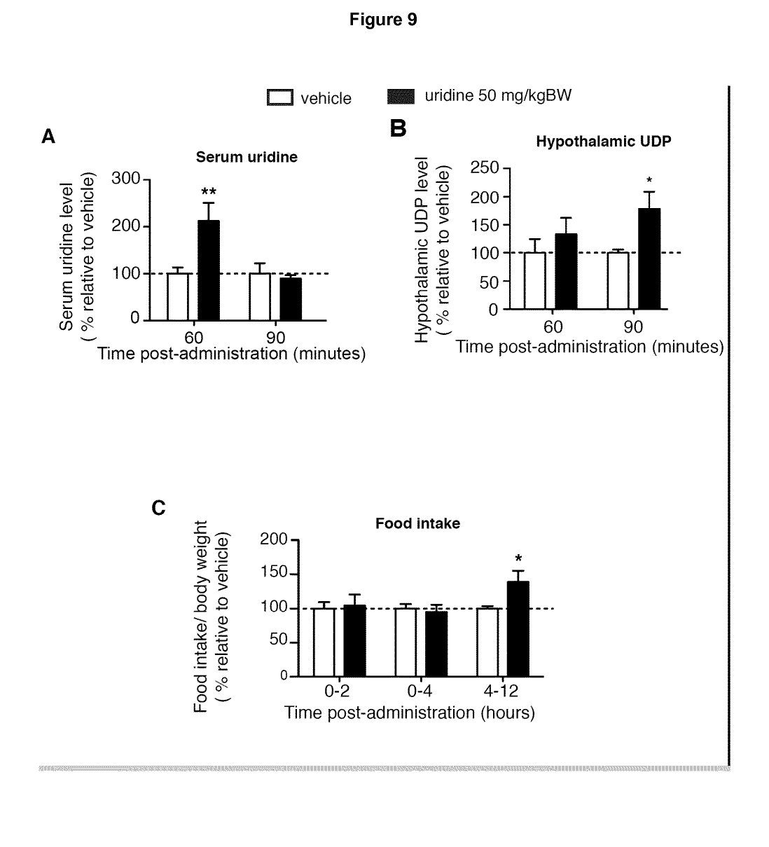

1. A method for treatment of diseases related to energy balance and carbohydrate metabolism and homeostasis comprising administering to a subject in need of treatment a therapeutically effective amount of at least one compound for inhibition of P2Y purinoceptor 6 polypeptide or for inactivation, degradation, downregulation or intercalation of a nucleic acid encoding P2Y purinoceptor 6.

2. The method according to claim 1, wherein the treatment of diseases related to energy balance and carbohydrate metabolism and homeostasis comprises:
a) inhibition of P2Y purinoceptor 6 polypeptide,
b) inactivation, degradation, downregulation or intercalation of a nucleic acid encoding P2Y purinoceptor 6,
c) downregulation of P2Y purinoceptor 6 signaling pathway,
d) inhibition of uridine transport across the blood brain barrier,
e) inhibition of UDP synthesis in the CNS, or
f) acceleration or increase of UDP degradation.

3. The method according to claim 1, wherein the disease related to energy balance and carbohydrate metabolism and homeostasis is selected from the group consisting of obesity, type 2 diabetes and related complications selected from cardiovascular diseases, hepatic steatosis, and lipid disorders.

4. The method according to claim 1, wherein the compound is
a) a small molecule,
b) an RNA molecule,
c) an siRNA molecule, an miRNA molecule, or a precursor thereof,
d) an antisense oligonucleotide,
e) an aptamer
f) a polypeptide,
g) an antibody, or
h) a ribozyme.

5. The method according to claim 1, wherein said compound is of general formula (I) 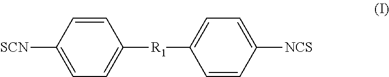
wherein
R₁ is selected from: trans-CH═CH—, —CH₂—CH₂—, —NHCSNH(CH₂)₂NHCSNH—, —NHCSNH(CH₂)₃NHCSNH—, and —NHCSNH(CH₂)₄NHCSNH—
or said compound is of general formula (II) 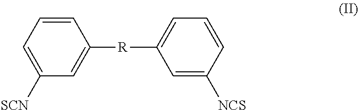
wherein
R is selected from: —NHCSNH(CH₂)₂NHCSNH—, —NHCSNH(CH₂)₃NHCSNH—, and —NHCSNH(CH₂)₄NHCSNH—,
or salts and solvates of compounds of general formula I and II.

6. The method according to claim 1, wherein said compound is selected from the group consisting of 1,4-di[3-(3-isothiocyanatophenyl)thioureido]butane, 1-isothiocyanato-4-[2-(4-isothiocyanato-phenyl)ethyl]benzene, and 1-amino-4-[[4-[[4-chloro-6-[(3-sulfophenyl)amino]-1,3,5-triazin-2-yl]amino]-3-sulfophenyl]amino]-9,10-dioxoanthracene-2-sulfonic acid.

7. A method for increasing the weight of a subject comprising administering to a subject in need of such treatment a therapeutically effective amount of at least one compound for:
a) activation of P2Y purinoceptor 6 polypeptide,
b) upregulation or modification for advanced transcriptional activity of a nucleic acid encoding P2Y purinoceptor 6,
c) upregulation of P2Y purinoceptor 6 signaling pathway,
d) activation of uridine transport across the blood brain barrier, or
e) activation or increase of UDP synthesis in the CNS.

8. The method according to claim 7, wherein the disease is selected from the group consisting of anorexia nervosa, cancer cachexia, and underweight.

9. The method according to claim 7, wherein the compound is
a) a small molecule,
b) an RNA molecule,
c) an siRNA molecule, an miRNA molecule, or a precursor thereof,
d) an antisense oligonucleotide,
e) an aptamer
f) a polypeptide,
g) an antibody, or
h) a ribozyme.

10. The method according to claim 7, wherein said compound is selected from the general formula (III) 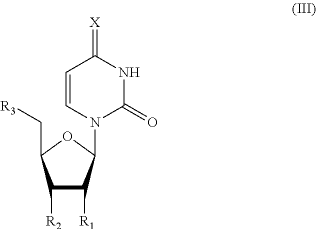
or salts and solvates thereof, wherein
X represents ═O or ═S,
R₁ represents —H, —OH, —OCHO, —OCOCH₃, —OCOC₂H₅, —OCOC₃H₇, —OCO-cyclo-C₃H₅, —OCOCH(CH₃)₂, —OCOC(CH₃)₃, —OCOC₄H₉, —OCOC₅H₁₁, —OCOCH(CH₃)—C₃H₇, —OCO—CH(CH₃)—C₂H₅, —OCOCH(CH₃)—CH(CH₃)₂, —OCOC(CH₃)₂—C₂H₅, —OCOCH₂—C(CH₃)₃, —OCO—C(CH₃)₃, —OCOCH(C₂H₅)₂, or —OCOC₂H₄—CH(CH₃)₂,
R₂ and R₃ represent independently of each other —OH, —OCHO, —OCOCH₃, —OCOC₂H₅, —OCOC₃H₇, —OCO-cyclo-C₃H₅, —OCOCH(CH₃)₂, —OCOC(CH₃)₃, —OCOC₄H₉, —OCOC₅H₁₁, —OCOCH(CH₃)—C₃H₇, —OCO—CH(CH₃)—C₂H₅, —OCOCH(CH₃)—CH(CH₃)₂, —OCOC(CH₃)₂—C₂H₅, —OCOCH₂—C(CH₃)₃, —OCO—C(CH₃)₃, —OCOCH(C₂H₅)₂, or —OCOC₂H₄—CH(CH₃)₂,
or the general formula (IV) 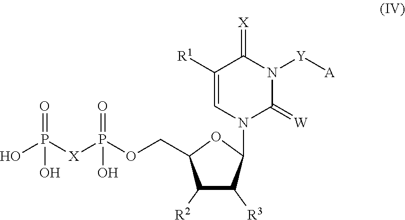
or salts and solvates thereof, wherein:
A is 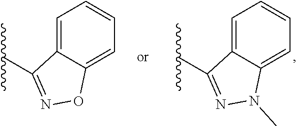 [SMILES: *c1noc2ccccc12 ; *c1nn(C)c2ccccc12]
and wherein A is optionally further substituted with one or more R⁷;
X is selected from —O—, —S—, —N(R⁵)—, —CH₂—, —C₂H₄—, —C₃H₆—, —C(CH₃)₂, and can independently and optionally be substituted with one or more R⁴;
Y is a bond, —CH₂—, —C₂H₄—, —C₃H₆—, —C(CH₃)₂—, —C₄H₅—, —CH₂—C(CH₃)₂—, —CH(CH₃)—C₂H₄—, —C₅H₁₀—, —CH(CH₃)—C₃H₆—, —CH₂—CH(CH₃)—C₂H₄—, —CH(CH₃)—CH(CH₃)—, —C(CH₃)₂—C₂H₄—, —CH₂—C(CH₃)₂—, —C(C₂H₅)₂—, or —C₂H₄—C(CH₃)₂—, and can independently and optionally be substituted with one or more R⁴;
Z and W are each independently selected from ═O, ═S, ═N(R⁵), and ═N—OR⁵;
R¹ is selected from: —H, halogen, —OR⁵, —CN, —CF₃, —OCF₃ and —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃, and can optionally be substituted with one or more R⁷;
R² and R³ are independently of each other selected from —OR⁵, —SR⁵, —NR⁵R⁶ and —OC(O)R⁵;
R⁴ is selected from: halogen, —OR⁵, —NO₂, —CN, —CF₃, —OCF₃, —R⁵, 1,2-methylenedioxy, 1,2-ethylenedioxy, —N(R⁵)₂, —SR⁵, —SOR⁵, —SO₂R⁵, —SO₂N(R⁵)₂, —SO₃R⁵, —C(O)R⁵, —C(O)C(O)R⁵, —C(O)CH₂C(O)R⁵, —C(S)R⁵, —C(S)OR⁵, —C(O)OR⁵, —C(O)C(O)OR⁵, —C(O)C(O)N(R⁵)₂, —OC(O)R⁵, —C(O)N(R⁵)₂, —OC(O)N(R⁵)₂, —C(S)N(R⁵)₂, —(CH₂)₀₋₂NHC(O)R⁵, —N(R⁵)N(R⁵)COR⁵, —N(R⁵)N(R⁵)C(O)OR⁵, —N(R⁵)N(R⁵)CON(R⁵)₂, —N(R⁵)SO₂R⁵, —N(R⁵)SO₂N(R⁵)₂, —N(R⁵)C(O)OR⁵, —N(R⁵)C(O)R⁵, —N(R⁵)C(S)R⁵, —N(R⁵)C(O)N(R⁵)₂, —N(R⁵)C(S)N(R⁵)₂, —N(COR⁵)COR⁵, —N(OR⁵)R⁵, —C(═NH)N(R⁵)₂, —C(O)N(OR⁵)R⁵, —C(═NOR⁵)R⁵, —OP(O)(OR⁵)₂, —P(O)(R⁵)₂, —P(O)(OR⁵)₂, and —P(O)(H)OR⁵);
R⁵ is selected from: —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃, 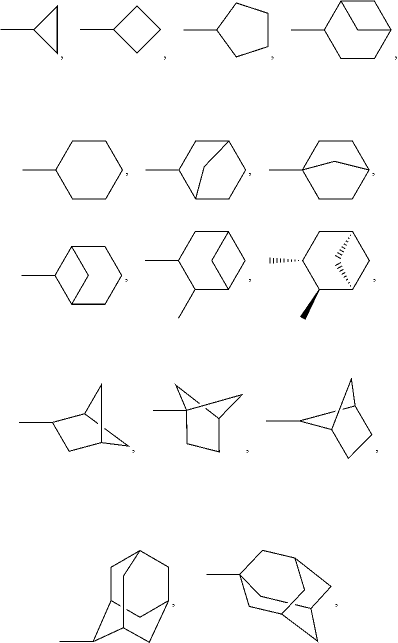
2-thienyl, 3-thienyl, 2-furanyl, 3-furanyl, 2-oxazolyl, 3-oxazolyl, 4-oxazolyl, 2-thiazolyl, 3-thiazolyl, 4-thiazolyl, 1-pyrazolyl, 3-pyrazolyl, 4-pyrazolyl, 5-pyrazolyl, 1-imidazolyl, 2-imidazolyl, 4-imidazolyl, 5-imidazolyl, phenyl, 1-naphthyl, 2-naphthyl, 2-pyridyl, 3-pyridyl, 4-pyridyl, 2-pyrimidinyl, 4-pyrimidinyl, 5-pyrimidinyl, 2-pyrazinyl, 3-pyridazinyl, 4-pyridazinyl, 1,3,5-triazin-2-yl, 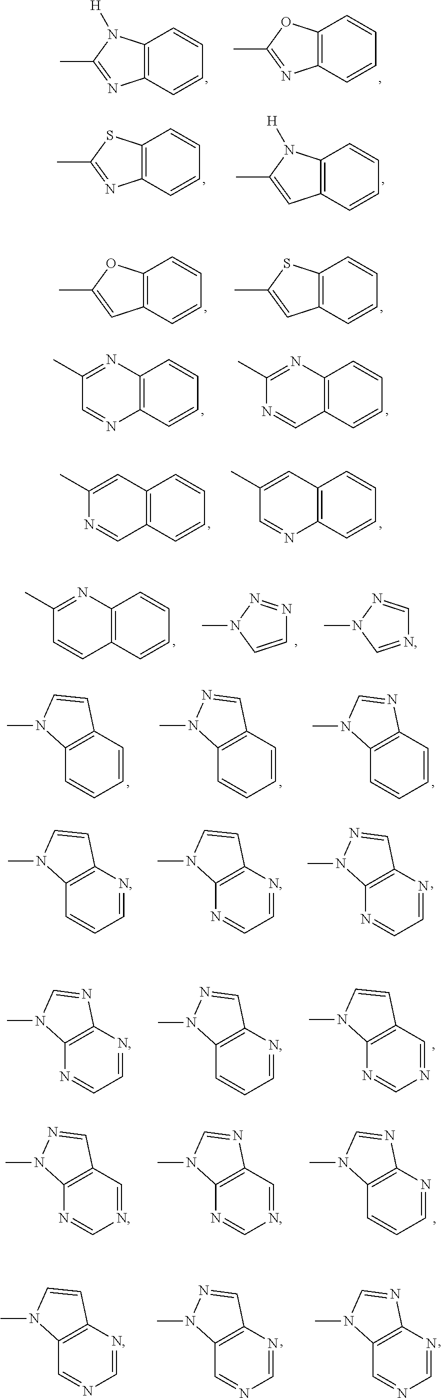 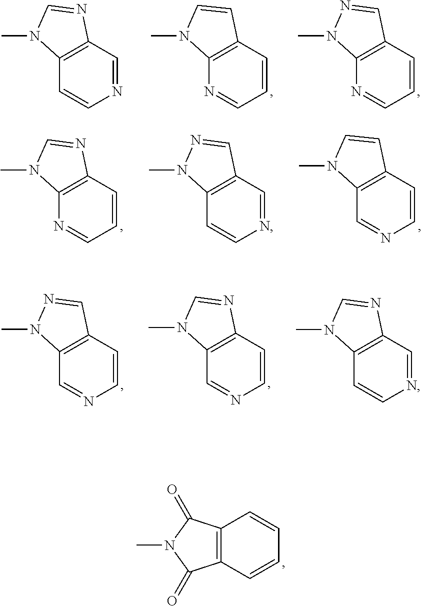
wherein two R⁵ groups bound to the same atom optionally form a 3- to 6-membered aromatic or non-aromatic ring having up to 3 heteroatoms independently selected from N, O, S, SO, or SO₂, wherein said ring is optionally fused to a (C6-C10)aryl, (C5-C10)heteroaryl, (C3-C10)cycloalkyl, or a (C3-C10)heterocycloyl;
R⁵ is independently and optionally substituted with one or more R⁷;
R⁶ is selected from: —R⁵, —C(O)R⁵, —C(O)OR⁵, —C(O)N(R⁵)₂ and —S(O)₂R⁵;
R⁷ is selected from: halogen, —OR⁸, —NO₂, —CN, —CF₃, —OCF₃, —R⁸, oxo, thioxo, 1,2-methylenedioxy, 1,2-ethylenedioxy, —N(R⁸)₂, —SR⁸, —SOR⁸, —SO₂R⁸, —SO₂N(R⁸)₂, —SO₃R⁸, —C(O)R⁸, —C(O)C(O)R⁸, —C(O)CH₂C(O)R⁸, —C(S)R⁸, —C(S)OR⁸, —C(O)OR⁸, —C(O)C(O)OR⁸, —C(O)C(O)N(R⁸)₂, —OC(O)R⁸, —C(O)N(R⁸)₂, —OC(O)N(R⁸)₂, —C(S)N(R⁸)₂, —(CH₂)₀₋₂NHC(O)R⁸, —N(R⁸)N(R⁸)COR⁸, —N(R⁸)N(R⁸)C(O)OR⁸, —N(R⁸)N(R⁸)CON(R⁸)₂, —N(R⁸)SO₂R⁸, —N(R⁸)SO₂N(R⁸)₂, —N(R⁸)C(O)OR⁸, —N(R⁸)C(O)R⁸, —N(R⁸)C(S)R⁸, —N(R⁸)C(O)N(R⁸)₂, —N(R⁸)C(S)N(R⁸)₂, —N(COR⁸)COR⁸, —N(OR⁸)R⁸, —C(═NH)N(R⁸)₂, —C(O)N(OR⁸)R⁸, —C(═NOR⁸)R⁸, —OP(O)(OR⁸)₂, —P(O)(R⁸)₂, —P(O)(OR⁸)₂, or —P(O)(H)(OR⁸); and
R⁸ is selected from: —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃.

11. The method according to claim 7, wherein said compound is a uridine precursor, uridine, uridine derivative, UDP or an activator of UDP synthesis, any regulator to increase UDP half-life or an activator of P2Y6 signaling pathway for use in a therapy for gaining or maintaining weight.

12. The method according to claim 7 for increasing the weight of an animal.

13. (canceled)

14. A method for screening for a compound for the treatment of diseases related to energy balance and carbohydrate metabolism and homeostasis, the method comprising
a) contacting a test compound with P2Y purinoceptor 6 polypeptide,
b) detecting the binding of said test compound to the P2Y purinoceptor 6 polypeptide, and
c) determining the activity of the P2Y purinoceptor 6 polypeptide in the presence of said test compound.

15. The method according to claim 14, wherein instead of polypeptides nucleic acids encoding the polypeptides are used and the expression rate is determined instead of the activity, preferably wherein the test compound is RNA or a peptide or an antibody or a small molecule.
